(19) United States
(12) Patent Application Publication
(10) Pub. No.: US 2023/0190676 A1
(43) Pub. Date: Jun. 22, 2023

(54) METHODS OF PROTECTING CELLS FROM INSULTS

(71) Applicant: MUSC FOUNDATION FOR RESEARCH DEVELOPMENT, Charleston, SC (US)

(72) Inventors: Michael Yost, Charleston, SC (US); Sarah Grace Dennis, Charleston, SC (US); Graham Warren, Charleston, SC (US)

(21) Appl. No.: 18/015,262

(22) PCT Filed: Jul. 9, 2021
(86) PCT No.: PCT/US2021/041172

§ 371 Date: Jan. 9, 2023

Related U.S. Application Data
(60) Provisional application No. 63/050,075, filed on Jul. 9, 2020.

(51) Int. Cl. A61K 31/08; A61P 39/00; A61N 5/10

(52) U.S. Cl. CPC A61K 31/08; A61P 39/00; A61N 5/10

(57) ABSTRACT

Described herein are methods for preventing non-insulted cell damage during a subject's exposure to radiation. In some embodiments, the method can include administering an amount of poloxamer 188 or a pharmaceutical formulation thereof to a subject prior to exposure to an insult.

### CROSS-REFERENCE TO RELATED APPLICATIONS

This application claims the benefit of and priority to co-pending U.S. Provisional Patent Application No. 63/050,075, filed on Jul. 9, 2020, entitled “METHODS OF PROTECTING CELLS FROM INSULTS,” the contents of which is incorporated by reference herein in its entirety.

### STATEMENT REGARDING FEDERALLY SPONSORED RESEARCH

This invention was made with government support under Grant No. OIA-1655740 awarded by National Science Foundation. The government has certain rights in the invention.

### TECHNICAL FIELD

The subject matter disclosed herein is generally directed to methods of protecting cells, such non-cancerous cells, from radiation and/or other insults.

### BACKGROUND

Radiotherapy is a well-established cancer treatment modality and has been in use for over fifty years. Radiation directly causes DNA damage to cells like single-strand breaks (SSBs), DSBs, DNA crosslink and DNA-Protein crosslinks, or induces damage indirectly to DNA by reactive oxygen species (ROS)/reactive nitrogen species (RNS), which leads to cell senescence and apoptosis. Radiation by nature does not discriminate between cancerous and non-cancerous cells. While methods and techniques of targeting only cancerous cells have been developed (e.g., sensitizing cancerous cells so that less of a radiation dose is needed and physically limiting the region(s) directly exposed), radiotherapy is still considered a “double-edged sword” and can still cause significant damage to non-cancerous cells.

Other insults, such as mechanical, chemical (e.g., hydrogen peroxide or other agent), or biological (e.g., viral and microorganisms), can result in ROS, RNS, peroxide, superoxide, or other inflammatory damage in the surrounding, in some cases remote, cells and tissue not directly exposed to the insult.

As such, there still exists a need for compositions, methods, and techniques for protecting adjacent cells and tissue not directly exposed to an insult that results in the production of ROS, RNS, peroxide, superoxide, or other inflammatory damage in the surrounding, in some cases remote, cells and tissues.

Citation or identification of any document in this application is not an admission that such a document is available as prior art to the present invention.

### SUMMARY

In certain example embodiments, described herein are methods of preventing damage to non-insulted cells in a subject during and/or after insult exposure comprising: administering a first amount of poloxamer 188 (P188) or a pharmaceutical formulation thereof to a subject prior to exposure of one or more regions of the subject to an insult; and exposing one or more regions of the subject to an insult after the blood concentration of P188 reaches between 1 and 5 mg/mL.

In certain example embodiments, the first amount of P188 is administered intravenously.

In certain example embodiments, the method further comprises administering an n^{th} amount of P188 or a pharmaceutical formulation thereof to the subject after administering the first amount of P188 and before exposing one or more regions of the subject to the insult, wherein n is 2 or more.

In certain example embodiments, the first amount, the n^{th} amount, or all of the amounts together of P188 is/are an amount effective to raise the blood concentration of P188 to 1 to 5 mg/mL within 1 to 48 hours.

In certain example embodiments, the first amount or the n^{th} amount of P188 is administered as a continuous infusion over a period of time.

In certain example embodiments, the first amount or one or more of the n^{th} amounts of P188 is administered as a bolus amount.

In certain example embodiments, only the first amount is administered and wherein the first amount is effective to raise the blood concentration of P188 to about 1 to about 5 mg/mL within 1 to 48 hours.

In certain example embodiments, the subject does not receive any amount of P188 for at least 14 hours after receiving the first or n^{th} amount of P188.

In certain example embodiments, P188 administration is discontinued immediately after exposing one or more regions of the subject to the insult.

In certain example embodiments, exposing one or more regions of the subject to the insult occurs about 1 to about 48 hours after administering the first or the n^{th} amount of P188.

In certain example embodiments, the insult is effective to kill one or more cancerous cells with in the one or more regions of the subject.

In certain example embodiments, the non-insulted cells are non-cancerous cells.

In certain example embodiments, the non-insulted cells are endothelial cells.

In certain example embodiments, the insult is a mechanical insult, a chemical insult, a biological insult, an energetic insult, or a combination thereof.

In certain example embodiments, the insult is ionizing radiation.

In certain example embodiments, described herein are methods of preventing damage to non-insulted cells in a subject during and/or after insult exposure comprising: administering an amount of poloxamer 188 (P188) or a pharmaceutical formulation thereof to a subject prior to exposure of one or more regions of the subject to an insult, wherein the amount ranges from about 10 to 150 mg/kg; and exposing one or more regions of the subject to the insult within 0-48 hours after administering the amount of P188 or pharmaceutical formulation thereof.

In certain example embodiments, the amount of P188 or pharmaceutical formulation thereof is administered intravenously.

In certain example embodiments, the administration of P188 or pharmaceutical formulation thereof is discontinued immediately prior to or immediately after exposing one or more regions of the subject to the insult.

In certain example embodiments, the subject does not receive any amount of P188 or pharmaceutical formulation thereof for at least 14 hours after administering the amount of P188 or pharmaceutical formulation thereof or after discontinuing the administration of P188 or pharmaceutical formulation thereof.

In certain example embodiments, the insult is a mechanical insult, a chemical insult, a biological insult, an energetic insult, or a combination thereof.

In certain example embodiments, the insult is ionizing radiation.

In certain example embodiments, described herein are kits that comprise an amount of P188 or a pharmaceutical formulation thereof, wherein the amount of P188 or pharmaceutical formulation is effective to prevent damage to non-insulted cells in a subject when one or more regions of the subject are exposed to an insult; and instructions fixed in a tangible medium of expression directing administration of the amount of the amount of P188 or pharmaceutical formulation thereof to a subject 0-48 hours prior to exposure of one or more regions of the subject to the insult.

In certain example embodiments, the insult is a mechanical insult, a chemical insult, a biological insult, an energetic insult, or a combination thereof.

In certain example embodiments, the insult is ionizing radiation.

In certain example embodiments, the instructions further direct for discontinuation of administration of the amount of P188 or pharmaceutical formulation thereof to the subject immediately prior to or immediately after exposure of one or more regions of the subject to the insult.

In certain example embodiments, the amount of P188 or pharmaceutical formulation thereof is effective to achieve a blood concentration of about 1 to about 5 mg/mL in the subject within 0-48 hours.

In certain example embodiments, described herein are methods of protecting non-insulted cells after exposure of a subject to an insult comprising: a) administering an amount of P188 or a formulation thereof to a subject prior to exposure to an insult; b) administering an amount of P188 or a formulation thereof to a subject immediately following exposure to an insult; c) administering an amount of P188 or a formulation thereof to a subject during an insult; or d) a combination thereof.

In certain example embodiments, the amount is effective increase the blood concentration of P188 in the subject to between 1 and 5 mg/mL.

In certain example embodiments, the amount is effective to coat one or more non-insulted cells.

In certain example embodiments, the amount is effective to reduce or prevent oxidative damage to one or more non-insulted cells.

In certain example embodiments, the amount is effective to reduce or prevent inflammatory damage to one or more non-insulted cells.

In certain example embodiments, administering occurs 0-48 hours prior to exposure to the insult.

In certain example embodiments, administering occurs 1-24 hours post exposure to the insult.

In certain example embodiments, the insult is a mechanical insult, a chemical insult, a biological insult, an energetic insult, or a combination thereof.

In certain example embodiments, the insult is ionizing radiation.

Described in certain example embodiments herein are methods of preventing insult-induced pneumonitis in a subject, the method comprising:
a) administering an amount of P188 or a formulation thereof to a subject prior to exposure to an insult;
b) administering an amount of P188 or a formulation thereof to a subject immediately following exposure to an insult;
c) administering an amount of P188 or a formulation thereof to a subject during an insult; or
d) a combination thereof.

In certain example embodiments, the amount is effective increase the blood concentration of P188 in the subject to between 1 mg/mL and 5 mg/mL.

In certain example embodiments, the amount is effective to coat one or more non-insulted cells.

In certain example embodiments, the amount is effective to reduce or prevent oxidative damage to one or more non-insulted cells.

In certain example embodiments, the amount is effective to reduce or prevent inflammatory damage to one or more non-insulted cells.

In certain example embodiments, administering occurs 0-48 hours prior to exposure to the insult.

In certain example embodiments, administering occurs 0-24 hours post exposure to the insult.

In certain example embodiments, the insult is a mechanical insult, a chemical insult, a biological insult, an energetic insult, or a combination thereof.

In certain example embodiments, the insult is ionizing radiation.

Described in certain example embodiments herein are methods of treating a disease or disorder in a subject in need thereof, the method including:
a) administering an amount of P188 or a formulation thereof to a subject prior to exposure to an insult;
b) administering an amount of P188 or a formulation thereof to a subject immediately following exposure to an insult;
c) administering an amount of P188 or a formulation thereof to a subject during an insult; or
d) a combination thereof.

In certain example embodiments, the amount is effective increase the blood concentration of P188 in the subject to between 1 mg/mL and 5 mg/mL.

In certain example embodiments, the amount is effective to coat one or more non-insulted cells.

In certain example embodiments, the amount is effective to reduce or prevent oxidative damage to one or more non-insulted cells.

In certain example embodiments, the amount is effective to reduce or prevent inflammatory damage to one or more non-insulted cells.

In certain example embodiments, administering occurs 0-48 hours prior to exposure to the insult. In certain example embodiments, administering occurs 0-24 or 0-48 hours post exposure to the insult. In certain example embodiments, P188 is not administered prior to and/or during insult exposure.

In certain example embodiments, the insult is a mechanical insult, a chemical insult, a biological insult, an energetic insult, a physiologic insult, or a combination thereof.

In certain example embodiments, the insult is an ischemic event, optionally a stroke or myocardial infarction.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows a panel of fluorescent microscopy images from in vitro studies that can demonstrate that Hiroloxamer protects endothelial cells from hydrogen peroxide.

FIGS. 2A-2B show graphs with result from in vitro studies that can demonstrate that Hiroloxamer does not affect cancer cell sensitivity to ionizing radiation as modeled by A549 (FIG. 2A) and H460 (FIG. 2B) human lung cancer cell lines.

FIGS. 3A-3P show fluorescent microscopy images from in vitro studies that demonstrate that Hiroloxamer preserves the actin cytoskeleton of human adipose microvascular endothelial cells directly exposed to ionizing radiation.

FIGS. 4A-4E show bright field images (FIGS. 4A-4D) and a graph (FIG. 4E) showing results from in vitro studies that can demonstrate viability of cells within an irradiated area (n=16).

FIGS. 5A-5E show bright field images (FIGS. 5A-5D) and a graph (FIG. 5E) showing results from in vitro studies that can demonstrate viability in the area adjacent to the irradiated area (n=9).

FIGS. 6A-6E show bright field images (FIGS. 6A-6D) and a graph (FIG. 6E) showing results from in vitro studies that can demonstrate viability in an area furthest away from the irradiated area (n=4).

FIGS. 7A-7L show fluorescent images showing results from in vitro studies that can demonstrate scaffold-free prevascularized endothelial-fibroblast constructs exposed to 10 Gy X-ray irradiation. FIGS. 7A-7D show Control, No Rx, No radiation (73.655 μm with 3.51 μm step size). FIGS. 7E-7H show Exposed to 10 Gy, No Rx (76.027 μm with 4 μm step size).

FIGS. 7I-7L show Rx pretreatment, exposed to 10 Gy (88.031 μm with 4 μm step size). Hoescht (nuclei), Phalloidin (F Actin), and CD31 (endothelial cell networks) are shown in respective greyscale.

FIGS. 8A-8C show several embodiments of a method of protecting cells, particularly non-cancerous cells, from radiation using a P188 formulation described herein.

FIGS. 9A-9C show fluorescent images showing results from in vitro studies that can demonstrate the effect of pretreatment with Hiorloxamer on F-actin and endothelial networks. SPEC Control: EC Networks are primitive, capillary-like networks. Control SPEC (6): 73.655 μm with 3.51 μm step size (22 steps). No Rx, XRT SPEC (3): 76.027 μm with 4 μm step size (28 steps). Rx Pretreat, Full Exposure SPEC (1): 88.031 μm with 4 μm step size (23 steps).

FIGS. 10A-10D show fluorescent images showing results from in vitro studies that can demonstrate the effect of pretreatment with Hiroloxamer on cells exposed to 1 Gy of radiation.

FIG. 11 shows fluorescent images showing results from in vitro studies that can demonstrate that 2 Gy exposure can lead to intracellular gap formation, which can be mitigated with pretreatment of Hiroloxamer. Stress fibers are required for inducing cell contraction, and dramatically influence the rate and size of the inter-endothelial gaps that form as cells retract from their borders (Pasain). Direct association of the actin cytoskeleton with cell adhesion proteins is essential to barrier function. Tight junctions and adherens junctions connect adjacent cells and regulate paracellular permeability. No Rx: Formation of intercellular gaps indicative of the loss of cell-cell junctions, which causes the depolymerization of F-actin in Ecs.

FIG. 12 shows show fluorescent images showing results from in vitro studies that can demonstrate the effect of pretreatment with Hiroloxamer on cells exposed to 50 Gy of radiation.

FIG. 13 shows lung histology showing results from in vivo studies of PBS or Hiroloxamer treated rats that can demonstrate the effect of 200 mg/kg Hiroloxamer treatment 3 hours prior to 20 Gy irradiation. Lung samples for histology were taken about 24 hours post irradiation.

FIG. 14 shows lung histology showing results from in vivo studies of PBS or Hiroloxamer treated rats that can demonstrate the effect of a 200 mg/kg Hiroloxamer treatment administered 15 min prior to exposure to 20 Gy of irradiation.

FIG. 15 shows lung histology showing results from in vivo studies of PBS or Hiroloxamer treated rats that can demonstrate the effect of a 200 mg/kg Hiroloxamer treatment administered 15 min prior to exposure to 20 Gy of irradiation. The histology reference of the healthy control was obtained from http://histology oucreate.com/Captions/Respiratory/109.ling.mammal/109.bronchiole.cl.40.L htm#click,

FIG. 16 shows lung histology showing results from in vivo studies of PBS or Hiroloxamer treated rats that can demonstrate the effect of a 200 mg/kg Hiroloxamer treatment administered 15 min prior to exposure to 20 Gy of irradiation.

FIG. 17 shows lung histology showing results from in vivo studies of PBS or Hiroloxamer treated rats that can demonstrate the effect of a 200 mg/kg Hiroloxamer treatment administered 15 min prior to exposure to 20 Gy of irradiation.

FIG. 18 shows lung histology showing results from in vivo studies of PBS or Hiroloxamer treated rats that can demonstrate that Hiroloxamer can conserve oxygen pathways in an irradiated lung at bronchiole branches. The histology reference of the healthy control was obtained from http://histology oucreate.com/Captions/Respiratory/109.ling.mammal/109.bronchiole.cl.40.L htm#click.

FIG. 19 shows lung histology showing results from in vivo studies of PBS or Hiroloxamer treated rats that can demonstrate the effect of a 200 mg/kg Hiroloxamer treatment administered 15 min prior to exposure to 20 Gy of irradiation.

FIG. 20 shows lung histology showing results from in vivo studies of PBS or Hiroloxamer treated rats that can demonstrate that Hiroloxamer attenuates inflammation of bronchioles via protection of the vasculature.

FIG. 21 shows lung histology showing results from in vivo studies of PBS and Hiroloxamer treated rats and a healthy control that can demonstrate that Hiroloxamer can conserve blood vessel (V) and bronchiole (B) architectures.

FIG. 22 shows lung histology showing results from in vivo studies of PBS and Hiroloxamer treated rats that can demonstrate that Hiroloxamer can conserve blood vessel (V) and bronchiole (B) architectures. FIG. 22 are higher magnification images of those shown in FIG. 21.

FIG. 23 shows lung histology showing results from in vivo studies of PBS and Hiroloxamer treated rats that can demonstrate that Hiroloxamer can conserve blood vessel (V) and bronchiole (B) architectures. Also shown is a healthy control.

FIG. 24 shows lung histology showing results from in vivo studies of PBS and Hirorloxamer that can demonstrate that Hiroloxamer can attenuate early radiation-induced damage in the lungs.

FIG. 25 shows lung histology showing results from in vivo studies of PBS and Hiroloxamer treated rats that can demonstrate that Hiroloxamer can conserve perivasculature of irradiated lungs.

FIG. 26 shows lung histology showing results from in vivo studies of PBS and Hiroloxamer treated rats that can demonstrate that Hiroloxamer can conserve perivasculature of irradiated lungs.

FIG. 27 shows lung histology showing results from in vivo studies of PBS and Hiroloxamer treated rats that can demonstrate that Hiroloxamer can conserve perivasculature of irradiated lungs. FIG. 27 is a higher magnification of the images shown in FIG. 26.

FIG. 28 shows an irradiation protocol for the results shown in FIGS. 13-27.

FIG. 29 shows a summary of the animal groups used in these experiments demonstrated in FIGS. 13-27.

FIG. 30 shows results showing results from an in vivo study 1 week post-irradiation (20 Gy). FIG. 30 shows a panel of representative photomicrographs of Hematoxylin & Eosin (H&E)-stained lung sections from Control, Radiation (RT), and Hiroloxamer+Radiation one week post-irradiation (Scale bar=250 μm). The control group had normal (clear) lumen of lung bronchi (“Br”) and normal lumen of air alveoli. The RT group had evident inflammatory infiltrate throughout the peribronchial, almost to occlusion, as well as collapsed blood vessels and capillaries, thickening of intra-alveolar septa, and cellular infiltrate surrounding bronchi structures. The Hiroloxamer+RT group had some minor inflammation around major vessels in the peribronchial, but alveoli and perivascular appeared normal.

FIG. 31 shows results showing results from an in vivo study 6 week post-irradiation (20 Gy) that can demonstrate that Hioloxamer can preserve healthy lung tissue in a partial volume rat lung X-ray irradiation model. FIG. 31 shows representative photomicrographs of Hematoxylin & Eosin (H&E)-stained right lung lower lobe sections from Control, Radiation (RT), and Hiroloxamer+Radiation six weeks post-irradiation (Scale bar=250 μm). The control group had normal (clear) lumen of lung bronchi (“Br”) and normal lumen of air alveoli. The RT group had marked interstitial edema, congested blood vessel and capillaries (“V”), increase in alveolar septal thickness, and dense inflammatory infiltrate throughout the entire lobe that expanded distally into the right upper lung lobe. The Hiroloxamer+RT group had some inflammatory infiltrate surrounding some bronchioles located near some minor alveoli thickening, blood vessels remained intact with minimal surrounding inflammatory infiltrate.

FIG. 32 shows representative photomicrographs of Picrosirius Red stained right lung lower lobe section from an in vivo study 6 weeks post-irradiation with 20Gy X-rays (scale bar=250 microns).

FIG. 33 shows representative photomicrographs of hematoxylin & Eosin (H&E)-stained right lung top lobe sections from Control, Radiation (RT), and Hiroloxamer+Radiation six weeks post-irradiation from an in vivo study (Scale bar=250 microns). Radiation-induced lung damage extended from the right lower lung lobe where radiation was exposed to the distal right upper lung lobe in the 20 Gy RT group only.

FIG. 34 shows representative images and photomicrographs that can demonstrate that Hiroloxamer prevents the onset of acute pneumonitis in a rat model of radiation-induced pneumonitis. Exposure of SD rats to a single fraction of 20Gy x-ray irradiation is a prescribed dose for radiation-induced pneumonitis (Ghita). Rats were anesthetized and restrained in a custom jig designed to expose only 6 mm of the right center lung lobe to irradiation, while the rest of the animal remained shielded through a lead shield. To investigate the onset and progression of pneumonitis, proximal and distal tissues to the radiation site were stained with Hematoxylin and Eosin (H&E) and evaluated to assess integrity of the major lung structures at 6 weeks post-irradiation. Representative photomicrographs of the right lung lower lobe sections from Healthy Normal, Vehicle (Saline)+RT, and Hiroloxamer+RT at 6-weeks post-irradiation. Vehicle (saline)+RT control animals developed acute pneumonitis within 6 weeks post-irradiation, as evidenced by capillary leak, marked interstitial edema, congested blood vessels and capillaries, increase in alveolar thickness, and dense inflammatory infiltrate that propagated distally to adjacent lung lobes and to the contralateral left lung. Animals treated with Hiroloxamer prior to irradiation (Hiroloxamer+RT) had clear lumen of peribronchial and bronchi with alveolar septum appearing normal as in the healthy control animals (Healthy Normal); n=3 for each group.

FIGS. 35A-35D shows representative photomicrographs (FIGS. 35A-35C) and a graph (FIG. 35D) that demonstrate an MPO Analysis of Leukocyte Infiltrate. Controlled neutrophil degranulation and myeloperoxidase (MPO) release at a site of damage is necessary for effective wound healing. However, over exuberant degranulation exaggerates the inflammatory response and can lead to tissue damage even in the absence of infection. Levels of MPO activity are indicative of the state of inflammation and oxidative stress in tissues. SD rats were irradiated as described previously with a single fraction of 20Gy X-ray to the right center lung lobe through a 6 mm hole in a lead shield to model radiation-induced pneumonitis. Sections from the three right lung lobes, as well as the contralateral left lung lobe were paraffin-embedded, sectioned to 5 μm, and stained with Hanker-Yates Peroxidase Leukocyte kit (Sigma Aldrich) for analyses. Stained sections were imaged using a LionHeartFX automated microscope, and MPO+ cell counts automated via thresholding in Gen5 software. Five high powered fields were analyzed for each animal with three animals per group. Representative MPO-stained right lower lung lobe sections of (FIG. 35A) Healthy control, (FIG. 35B) vehicle (Saline)+RT, (FIG. 35C) 200 mg/kg Hiroloxamer+RT. Tissue damage was observed in the control saline+RT animals, that had significantly elevated levels of MPO in all lung lobes compared to the Hiroloxamer+RT and healthy normal (FIG. 35D). Statistical comparison (ANOVA followed by Tukey) of healthy normal, vehicle (Saline)+RT, and Hiroloxamer+RT shows no difference between healthy normal and Hiroloxamer+RT, while there is a significant statistical difference between vehicle (Saline)+RT and healthy normal (p<0.001), and vehicle (Saline)+RT and Hiroloxamer+RT (p<0.001).

FIGS. 36A-36B show graphs that demonstrate radiotherapy effectively kills human cancer cells in combination with dose-escalated Hiroloxamer. A549 and H460 human cancer cell lines were treated with Hiroloxamer prior to a single fraction of irradiation with 0, 2, or 6Gy. No significant survival trends were observed with dose-escalated Hiroloxamer treatment.

FIG. 37 shows representative photomicrographic images that can demonstrate that Hiroloxamer protects healthy tissue from radiation toxicity independent of irradiation dose. Animals were exposed to either 10Gy×1 fraction (fx), 20Gy×1fx, or 10Gy×4fx of X-ray irradiation delivered every 2-3 days over 2 weeks, to an 8 mm area of the right lung. All animals were sacrificed at 6-weeks post-irradiation and stained with H&E (5 μm sections). Animals treated with vehicle (top row of figures) had evident radiation-induced lung injury of varying degrees based on dose. 20Gy×1fx had the most significant damage, evidenced by edema, inflammatory infiltrate, and congestion of the lung parenchyma that were not localized to the irradiated but spread to all lung lobes bilaterally. These histological findings are indicative of the onset of radiation-induced acute pneumonitis. 10Gy×1fx and 10Gy×4fx had less damage than the 20Gy×1fx, which aligns with the classical theory that healthy tissue toxicity is dependent on the dose per fraction rather than the total dose delivered. Animals pre-treated with 200 mg/kg Hiroloxamer only had damage localized to the targeted irradiated area, with healthy tissue protected from toxicities in all radiotherapy regimens assessed.

FIGS. 38A-38D shows representative photomicrographic images that can demonstrate that Hiroloxamer protects healthy tissue from radiation toxicity in a dose-dependent manner. Animals were exposed to 10Gy×4 fractions of X-ray irradiation, delivered every 2-3 days over 2 weeks, to an 8 mm area of the right lung. Animals treated with 200 mg/kg Hiroloxamer (FIG. 38D) prior to irradiation had normal lung appearance comparable to healthy control (FIG. 38A). Animals treated with 50 mg/kg Hiroloxamer (FIG. 38C) had evident cellular infiltrate and subsequent thickening of the alveoli that was not as severe as the vehicle (PBS)-treated animals (FIG. 38B), but not adequate protection compared to the 200 mg/kg dose.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further embodiment includes from the one particular value and/or to the other particular value. The recitation of numerical ranges by endpoints includes all numbers and fractions subsumed within the respective ranges, as well as the recited endpoints. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g., given data set, art accepted standard, and/or with e.g., a given confidence interval (e.g., 90%, 95%, or more confidence interval from the mean), such as variations of +1-10% or less, +/−5% or less, +/−1% or less, and +1-0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, a “biological sample” is a sample that contains whole cells and/or live cells, and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

As used herein, “administering” refers to any suitable administration for the agent(s) being delivered and/or subject receiving said agent(s) and can be oral, topical, intravenous, subcutaneous, transcutaneous, transdermal, intramuscular, intra joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g., by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

As used herein “cancer” refers to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, Hairy cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer; uterine sarcoma, Sézary syndrome, skin cancer, small intestine cancer, large intestine cancer (colon cancer); soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “chemotherapeutic agent” or “chemotherapeutic” refers to a therapeutic agent utilized to prevent or treat cancer.

As used herein, “control” is a term of art and refers to an alternative subject or sample used in an experiment for comparison purpose and included to minimize or distinguish the effect of variables other than an independent variable. Controls can be positive or negative. Use of suitable controls to determine a particular effect or lack of a measurable or observable effect will be appreciated by those of ordinary skill in the art.

As used herein, the terms “disease” or “disorder” are used interchangeably throughout this specification and refer to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, disorder, sickness, illness, complaint, indisposition, or affliction.

As used herein, “dose,” “unit dose,” or “dosage” refers to physically discrete units suitable for use in a subject, each unit containing a predetermined quantity of the Hiroloxamer and/or a pharmaceutical formulation thereof calculated to produce the desired response or responses in association with its administration.

As used herein, “Hiroloxamer” refers to a long circulating material free poloxamer 188 (P188) composition that has been purified such that the polydispersity is less than about 1.07 and is as described in U.S. Pat. No. 9,403,941.

The term “molecular weight”, as used herein generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “optional” or “optionally” means that the subsequent described event, circumstance or substituent may or may not occur, and that the description includes instances where the event or circumstance occurs and instances where it does not.

As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo.

As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient.

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic.

As used herein, “preventative”, “prevention”, “prevent”, “prophylactic”, and the like refer to hindering or stopping (by action of a compound, formulation, and/or method) a disease or condition before it occurs, even if undiagnosed, or while the disease or condition is still in the sub-clinical phase.

As used herein, the term “radiation sensitizer” refers to agents that can selectively enhance the cell killing from irradiation in a desired cell population, such as tumor cells, while exhibiting no single agent toxicity on tumor or normal cells.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed by the term “subject”.

As used herein, “substantially pure” means an object species is the predominant species present (i.e., on a molar basis it is more abundant than any other individual species in the composition), and preferably a substantially purified fraction is a composition wherein the object species comprises about 50 percent of all species present. Generally, a substantially pure composition will comprise more than about 80 percent of all species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single species.

As used interchangeably herein, the terms “sufficient” and “effective,” refer to an amount (e.g., mass, volume, dosage, concentration, and/or time period) needed to achieve one or more desired and/or stated result(s). For example, a therapeutically effective amount refers to an amount needed to achieve one or more therapeutic effects.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, “therapeutic” refers to treating, healing, and/or ameliorating a disease, disorder, condition, or side effect, or to decreasing in the rate of advancement of a disease, disorder, condition, or side effect. A “therapeutically effective amount” can therefore refer to an amount of a compound that can yield a therapeutic effect.

As used herein, the terms “treating”, and “treatment” refer generally to obtaining a desired pharmacological and/or physiological effect. The effect can be, but does not necessarily have to be, prophylactic in terms of preventing or partially preventing a disease, symptom or condition thereof, such as cancer and/or indirect radiation damage. The effect can be therapeutic in terms of a partial or complete cure of a disease, condition, symptom or adverse effect attributed to the disease, disorder, or condition. The term “treatment” as used herein covers any treatment of cancer and/or indirect radiation damage, in a subject, particularly a human and/or companion animal, and can include any one or more of the following: (a) preventing the disease or damage from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic (preventative) treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

As used herein, “water-soluble”, generally means at least about 10 g of a substance is soluble in 1 L of water, i.e., at neutral pH, at 25° C.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Radiotherapy is a well-established cancer treatment modality and has been in use for over fifty years. Radiation is also round in other areas, such as nuclear power and in research. Thus, there is potential for radiation exposure both intentional (e.g., therapeutic purposes), and accidental or unintended (such as in a radiation spill or exposure to a contaminated environment) Radiation (no matter the source) directly causes DNA damage to cells like single-strand breaks (SSBs), DSBs, DNA crosslink and DNA-Protein crosslinks or induces damage indirectly to DNA by reactive oxygen species (ROS)/reactive nitrogen species (RNS), which leads to cell senescence and apoptosis. Radiation by nature does not discriminate between cancerous and non-cancerous cells. While methods and techniques of targeting radiation in a therapeutic context to only cancerous or specific types of cells or tissue have been developed (e.g., sensitizing cancerous cells so that less of a radiation dose is needed and physically limiting the region(s) directly exposed), radiotherapy is still considered a “double-edged sword” and can still cause significant damage to non-cancerous cells. Further, accidental radiation exposure presents a significant risk to health as in most cases precautions to limit exposure are often not taken or are inadequate as exposure was not planned for. Other insults, such as mechanical, chemical (e.g., hydrogen peroxide or other agents), or biological (e.g., viral and microorganisms), can result in ROS, RNS, peroxide, superoxide, or other inflammatory damage in the surrounding, in some cases remote, cells and tissue not directly exposed to the insult.

Despite the vast benefits of radiotherapies and diagnostics, ionizing and other types of radiation can be harmful to otherwise normal and healthy cells. See e.g., Desouky et al., 2015, J. Rad Res App Sci. 8(2):247-254. Ionizing radiation is energetic and penetrating and has direct and indirect effects. In the direct action, radiation hits a DNA molecule directly and alters its molecular structure, which can lead to cell damage and even cell death. Surviving cells with damage may later become cancerous or harbor other abnormalities that impair normal functionality. In indirect action, the radiation hits the water molecules and other organic molecules inside or outside of the cell, whereby free radicals such as hydroxyl and alkoxy moieties are produced. Reactive nitrogen species can also be produced by direct ionization of DNA, which can also have detrimental indirect effects.

Also, radiation can induce what are referred to in the field as “bystander effects”. Ionizing radiation-induced bystander effects can be defined as the occurrence of biological effects in un-irradiated cells as a result of exposure of other cells in the population to radiation. These bystander effects appear more pronounced with low doses of radiation. Thus, current efforts to make radiotherapy safer that employ using less radiation may be actually increasing the incidence of deleterious bystander effects in healthy non-cancerous cells. The bystander effect is believed to result from a bystander signal that can be transmitted through direct cell-to-cell contact or through soluble factors (e.g., reactive oxygen and reactive nitrogen species, cytokines, calcium ions, and small RNAs) released into the environment and circulation (see e.g., J. B. Little. 2006, Mut. Res. Fund. Molc. Mech. Mutagen. 597:113-118). See also Williams, J. P. McBride, W. H., 2011. Int J Radiat Biol 87(8):851-868.

In similar fashion and without being bound by theory, the ‘bystander effect’ observed in the context of radiation exposure can also occur secondary to mechanical, chemical, and/or biological insult resulting in ROS, RNS, peroxide, superoxide, and/or inflammatory responses. As such, there still exists a need for compositions, methods, and techniques for protecting cells and tissue not directly exposed to an insult that results in the production of ROS, RNS, peroxide, superoxide, or other inflammatory damage in the surrounding, in some cases remote, cells and tissues.

With that said, embodiments disclosed herein can provide methods to protect non-insulted cells, during and/or after exposure of cells to an insult. As used herein “non-insulted cells” refers to adjacent and/or remote cells to insulted cells and are cells that have not been directly exposed to an insult, which can be mechanical, chemical, biological, or energetic. It will be appreciated that insults cause a negative change and/or damage to the cell which it directly impacts. The insult can result in the product of free-radicals (e.g., ROS, RNS, peroxide, superoxide) and/or inflammation from the insulted cells and/or non-insulted cells. As previously discussed, cells adjacent to and remote can be indirectly damaged though, among other mechanisms, the ‘bystander effect’. In some embodiments, the method can include administering a first amount of poloxamer 188 (P188) or a pharmaceutical formulation thereof to a subject prior to exposure of one or more regions of the subject to an insult; and exposing (on purpose (for treatment) or accidently) one or more regions of the subject to an insult after the blood concentration of P188 reaches between about land 5 mg/mL, e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL.

In some embodiments, the method can include administering a first amount of poloxamer 188 (P188) or a pharmaceutical formulation thereof to a subject about 0 hours (i.e., at the time of insult)-48 hours prior to exposure of one or more regions of the subject to an insult; and exposing (either on purpose (e.g., for treatment) or accidently) one or more regions of the subject to an insult.

Also provided herein are kits that can contain an effective amount of P188 or a pharmaceutical formulation thereof and instructions to administer 0-48 hours prior to exposure of or more regions of a subject to ionizing radiation. In some embodiments, the methods can prevent cell damage in on or more non-insulted cells proximal and/or remote to the insulted cells, such as in a subject that has been exposed to an insult.

Embodiments disclosed herein can provide methods to protect cells, particularly non-cancerous cells and/or non-irradiated cells, during and/or after radiation exposure such as that which occurs during radiotherapy for cancer. In some embodiments, the method can include administering a first amount of poloxamer 188 (P188) or a pharmaceutical formulation thereof to a subject prior to exposure of one or more regions of the subject to ionizing radiation; and exposing one or more regions of the subject to ionizing radiation after the blood concentration of P188 reaches between about land 5 mg/mL, e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL.

In some embodiments, the method can include administering a first amount of poloxamer 188 (P188) or a pharmaceutical formulation thereof to a subject about 0-48 hours prior to exposure of one or more regions of the subject to ionizing radiation; and exposing one or more regions of the subject to ionizing radiation. Also provided herein are kits that can contain an effective amount of P188 or a pharmaceutical formulation thereof and instructions to administer 0-48 hours prior to exposure of or more regions of a subject to ionizing radiation. In some embodiments, the methods can prevent cell damage, such as non-cancerous and/or non-irradiated cell damage in a subject that has been exposed to ionizing radiation.

Without being bound by theory, the poloxamer 188 formulations described herein can be capable of coating cells as opposed to being sequestered in the blood and can provide protection from free radicals, DAMPs, and components of an inflammatory cascade. Free radicals, DAMPS, and components of an inflammatory cascade can be the result of or a product produced from an insulted cell that is adjacent to or remote from a non-insulted cell and cause damage to the non-insulted cell. The compositions and methods described herein can have the advantage of e.g., protecting undamaged cells, repairing damaged cells, and/or attenuating insult- or inflammatory-process by sparing cells and/or tissue that would otherwise be lost due to the insult and various downstream effects. See also Goliaei et al., 2017. J Phys Chem B. 120(33)8361-8641. It is further believed that embodiments of the P188 formulation described herein involves hydrophobic-hydrophilic associative interaction with cell surface lipid bilayer membranes. Once on the cell surface, the polymer forms a network of intermolecular hydrogen bonds between polymer molecules, and between polymer molecules and water. This protective layer scavenges and quenches free radicals and closes damage-associated pores within the cell membrane by increasing the packing density of the lipid bilayer, and can lead to one or more of the effects state herein. This can allow the cell to absorb and/or neutralize free radicals. This can close holes in cell membranes and repair damage portions of the cell membrane. This can minimize the release of inflammatory molecules and damage-associated molecular patterns. This can also provide blood rheological modification that can involve associated with red blood cells and vascular endothelium to lower the viscosity and cell-cell interactions. In some embodiments, the purified forms of the P188 (including, but not limited to Hiroloxamer), can result in these specific mechanism of actions.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Poloxamer Formulations

Described herein are pharmaceutical formulations that can contain an amount, effective amount, and/or least effective amount, and/or therapeutically effective amount of a suitable poloxamer, e.g., P188 (which is also referred to as the primary active agent or ingredient elsewhere herein) and a pharmaceutically acceptable carrier. The P188 pharmaceutical formulation can then be administered to a subject in need thereof according to a method described elsewhere herein.

Poloxamers are synthetic tri-block copolymers composed of a central hydrophobic chain of polyoxypropylene flanked by two hydrophilic chains of polyoxyethylene with a weight ratio of 4:2:4 and are according to Formula I where a=80 and b=27.

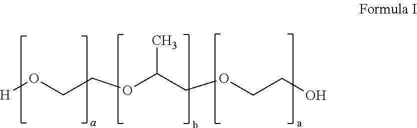

This arrangement results in an amphiphilic surface copolymer whose molecular size, hydrophilicity and hydrophobicity can by varied by altering the number of central and side chains of the molecule. Poloxamers are commonly abbreviated with a letter “P” (for poloxamer) followed by three digits. The first two digits multiplied by 100 give the approximate molecular mass of the polyoxypropylene core and the last digit multiplied by 10 gives the percentage of polyoxyethylene content. Poloxamer 188 (P188) is a nonionic linear copolymer having an average molecular weight of 8400 Daltons. It is known commercially as PLURONIC F68, FLOCOR, and RheothRx. P188 is also described in U.S. Pat. No. 5,696,298.

In some embodiments, P188 can be chemically synthesized via a two-step process. In the first step building the (poly)oxypropylene core, and second by addition of poly(oxyethylene) to the terminal ends of the polyoxypropylene core. Because of variation in the rates of polymerization during both steps, synthesized P188 contains a bell-shaped distribution of polymer species, which vary primarily in overall chain length. In some embodiments, the P188 can be filtered to reduce the polydispersity to produce a purified P188 suitable for use in embodiments of the methods described herein. In some embodiments, the low molecular weight products present are removed. In some embodiments, both the low and the high molecular weight products present are removed by any suitable size fractionation or separation technique.

In some embodiments, the pharmaceutical formulation contains an amount of a purified P188, such that the P188 formulation contains substantially no low molecular weight products (i.e., products with a molecular weight of about 5,500 Da or less). In some embodiments, the formulation contains a purified P188 such that the formulation contains substantially no low molecular weight products and substantially no high molecular weight products (i.e., those products with a molecular weight of about 16,000 Da or greater). In some embodiments, the P188 formulation described herein contains P188 molecules having a molecular weight ranging between 7,000 Da to about 15,500 Da. In some embodiments, the average molecular weight of P188 molecules is about 8.740. In some embodiments, the average molecular weight of the P188 is about 8, 740, with molecules ranging in size from about 7,000 Da to about 15,500 Da. In some embodiments, the polydispersity is about 1.07 or less, e.g., (1.06, 1.05, 1.04, 1.03, 1.02, 1.01 or 1).

In some embodiments, the P188 is Hiroloxamer. In some embodiments, the P188 is a P188 composition as described in U.S. Pat. No. 9,403,941.

In some embodiments, the pharmaceutical formulation contains an amount of Hiroloxamer.

### Pharmaceutically Acceptable Carriers and Auxiliary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with auxiliary agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of auxiliary active agents, including but not limited to, biologic agents or molecules (including but not limited to (e.g.; polypeptides, polynucleotides, antibodies and fragments thereof, aptamers, and the like), chemotherapeutics, antineoplastic agents, hormones, antibiotics, antivirals, immunomodulating agents, antinausea, pain modifying compounds (such as opiates), anti-inflammatory agents, antipyretics, antibiotics, and combinations thereof.

### Effective Amounts

In some embodiments, the amount of the primary active agent (e.g., P188 or purified P188) and/or optional auxiliary active agent can be an effective amount, least effective amount, and/or therapeutically effective amount. The effective amount, least effective amount, and/or therapeutically effective amount of the primary and/or optional auxiliary active agent described elsewhere herein contained in the pharmaceutical formulation can be a non-zero amount ranging from about 0 or about 1 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or to 1000 pg, ng, μg, mg, or g or be any numerical value with any of these ranges. In some embodiments, the effective amount, least effective amount, and/or therapeutically effective amount can be an effective concentration, least effective concentration, and/or therapeutically effective concentration, each of which can be a non-zero amount ranging from about 0 or about 1 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or to 1000 pM, nM, μM, mM, or M or be any numerical value with any of these ranges.

In other embodiments, the effective amount, least effective amount, and/or therapeutically effective amount of an auxiliary active agent present in the formulation can be a non-zero amount ranging from about 0 or about 1 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or to 1000 IU or be any numerical value with any of these ranges.

In some embodiments, a primary active agent is present in the pharmaceutical formulation and can be a non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% or to about 100% w/w, v/v, or w/v of the pharmaceutical formulation.

In some embodiments, the auxiliary active agent, when optionally present, can be a non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9%, or to about 100% w/w, v/v, or w/v of the pharmaceutical formulation.

In embodiments where there is an auxiliary active agent contained in the pharmaceutical formulation, the effective amount of the auxiliary active agent will vary depending on the auxiliary active agent.

When optionally present in the pharmaceutical formulation, the auxiliary active agent can be included in the pharmaceutical formulation or can exist as a stand-alone compound or pharmaceutical formulation that can be administered contemporaneously or sequentially with the compound, derivative thereof, or pharmaceutical formulation thereof. In yet other embodiments, the effective amount of the auxiliary active agent can be a non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, to about 99.9% w/w, v/v, or w/v of the total auxiliary active agent pharmaceutical formulation. In additional embodiments, the effective amount of the auxiliary active agent can be anon-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or to about 99.9% w/w, v/v, or w/v of the total pharmaceutical formulation.

### Dosage Forms

In some embodiments, the pharmaceutical formulations described herein can be in a dosage form. The dosage form can be administered to a subject in need thereof. The dosage form can be effective generate specific concentration, such as an effective concentration, at a given site in the subject in need thereof. In some cases, the dosage form contains a greater amount of the active ingredient or auxiliary active ingredient than the final intended amount needed to reach a specific region or location within the subject.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional auxiliary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, Md., 2000, and “Pharmaceutical dosage forms and drug delivery systems”, 6th Edition, Ansel et al., (Media, Pa.: Williams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional auxiliary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional auxiliary active ingredient, and/or pharmaceutically acceptable salt thereof where appropriate can be formulated with a paraffinic or water-miscible ointment base. In other embodiments, the primary and/or auxiliary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional auxiliary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or auxiliary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, auxiliary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional auxiliary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, or 3 doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional auxiliary active ingredient, and/or pharmaceutically acceptable salt thereof where appropriate, such a dosage form can contain a powder base such as lactose, glucose, trehalose, mannitol, and/or starch. In some of these embodiments, a primary active agent, auxiliary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, auxiliary active ingredient, and/or pharmaceutically acceptable salt thereof where appropriate per unit dose. In an embodiment, the predetermined amount of primary active agent, auxiliary active ingredient, and/or pharmaceutically acceptable salt thereof where appropriate can be an effective amount, a least effect amount, and/or a therapeutically effective amount. In some embodiments, the predetermined amount can be effective to protect non-irradiated cells when one or more regions of the subject are exposed to ionizing radiation.

In other embodiments, the predetermined amount of a primary active agent, auxiliary active agent, and/or pharmaceutically acceptable salt thereof where appropriate, can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies, auxiliary agents, and/or multiple pharmaceutical formulations are to be delivered to a subject and/or a cell, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time. In some embodiments, the auxiliary agent, co-therapy, and the like can be administered before, after or both before and after the poloxamer formulation.

### Exemplary Auxiliary Active Agents/Co-Therapies

In some embodiments as previously discussed, one or more additional compounds can be administered as an auxiliary active agents. Such auxiliary active agents can include, but are not limited to, polynucleotides, amino acids, peptides, polypeptides, antibodies, aptamers, ribozymes, guide sequences for ribozymes that inhibit translation or transcription of essential tumor proteins and genes, hormones; immunomodulators, antipyretics, anxiolytics, antipsychotics, analgesics, antispasmodics, anti-inflammatories, anti-histamines, anti-infectives; chemotherapeutics, radiation sensitizers, and combinations thereof.

Suitable radiation sensitizers include, but are not limited to, 5-fluorouracil, platinum analogs (e.g., cisplatin, carboplatin, and oxaliplatin), gemcitabine, DNA topoisomerase I-targeting drugs (e.g. camptothecin derivatives (e.g., topotecan and irinotecan)), epidermal growth factor receptor blockade family agents (e.g., cetuximab, gefitinib), farnesyltransferase inhibitors (e.g., L-778-123), COX-2 inhibitors (e.g., rofecoxib, celecoxib, and etoricoxib), bFGF and VEGF targeting agents (e.g., bevazucimab and thalidomide), NBTXR3, Nimoral, trans sodium crocetinate, NVX-108, and combinations thereof. See also e.g., Kvols, L. K., J Nucl Med 2005; 46:187S-190S.

Suitable hormones include, but are not limited to, amino-acid derived hormones (e.g., melatonin and thyroxine), small peptide hormones and protein hormones (e.g., thyrotropin-releasing hormone, vasopressin, insulin, growth hormone, luteinizing hormone, follicle-stimulating hormone, and thyroid-stimulating hormone), eicosanoids (e.g., arachidonic acid, lipoxins, and prostaglandins), and steroid hormones (e.g., estradiol, testosterone, tetrahydro testosterone, cortisol).

Suitable immunomodulators include, but are not limited to, prednisone, azathioprine, 6-MP, cyclosporine, tacrolimus, methotrexate, interleukins (e.g., IL-2, IL-7; and IL-12), cytokines (e.g., interferons (e.g., IFN-α, IFN-β, IFN-ε, IFN-K, IFN-ω, and IFN-γ), granulocyte colony-stimulating factor, and imiquimod), chemokines (e.g., CCL3, CCL26 and CXCL7), cytosine phosphate-guanosine, oligodeoxynucleotides, glucans, antibodies, and aptamers).

Suitable antipyretics include, but are not limited to, non-steroidal anti-inflammatories (e.g., ibuprofen, naproxen, ketoprofen, and nimesulide), aspirin and related salicylates (e.g., choline salicylate, magnesium salicylate, and sodium salicylate), paracetamol/acetaminophen, metamizole, nabumetone, phenazone, and quinine.

Suitable anxiolytics include, but are not limited to, benzodiazepines (e.g., alprazolam, bromazepam, chlordiazepoxide, clonazepam, clorazepate, diazepam, flurazepam, lorazepam, oxazepam, temazepam, triazolam, and tofisopam), serotonergic antidepressants (e.g., selective serotonin reuptake inhibitors, tricyclic antidepressants, and monoamine oxidase inhibitors), mebicar, fabomotizole, selank, bromantane, emoxypine, azapirones, barbiturates, hydroxyzine, pregabalin, validol, and beta blockers.

Suitable antipsychotics include, but are not limited to, benperidol, bromoperidol, droperidol, haloperidol, moperone, pipamperone, timiperone, fluspirilene, penfluridol, pimozide, acepromazine, chlorpromazine, cyamemazine, dixyrazine, fluphenazine, levomepromazine, mesoridazine, perazine, pericyazine, perphenazine, pipotiazine, prochlorperazine, promazine, promethazine, prothipendyl, thioproperazine, thioridazine, trifluoperazine, triflupromazine, chlorprothixene, clopenthixol, flupentixol, thiothixene, zuclopenthixol, clotiapine, loxapine, prothipendyl, carpipramine, clocapramine, molindone, mosapramine, sulpiride, veralipride, amisulpride, amoxapine, aripiprazole, asenapine, clozapine, blonanserin, iloperidone, lurasidone, melperone, nemonapride, olanzapine, paliperidone, perospirone, quetiapine, remoxipride, risperidone, sertindole, trimipramine, ziprasidone, zotepine, alstonie, bifeprunox, bitopertin, brexpiprazole, cannabidiol, cariprazine, pimavanserin, pomaglumetad methionil, vabicaserin, xanomeline, and zicronapine.

Suitable analgesics include, but are not limited to, paracetamol/acetaminophen, nonsteroidal anti-inflammatories (e.g., ibuprofen, naproxen, ketoprofen, and nimesulide), COX-2 inhibitors (e.g., rofecoxib, celecoxib, and etoricoxib), opioids (e.g., morphine, codeine, oxycodone, hydrocodone, dihydromorphine, pethidine, buprenorphine), tramadol, norepinephrine, flupirtine, nefopam, orphenadrine, pregabalin, gabapentin, cyclobenzaprine, scopolamine, methadone, ketobemidone, piritramide, and aspirin and related salicylates (e.g., choline salicylate, magnesium salicylate, and sodium salicylate).

Suitable antispasmodics include, but are not limited to, mebeverine, papaverine, cyclobenzaprine, carisoprodol, orphenadrine, tizanidine, metaxalone, methocarbamol, chlorzoxazone, baclofen, dantrolene, baclofen, tizanidine, and dantrolene. Suitable anti-inflammatories include, but are not limited to, prednisone, non-steroidal anti-inflammatories (e.g., ibuprofen, naproxen, ketoprofen, and nimesulide), COX-2 inhibitors (e.g., rofecoxib, celecoxib, and etoricoxib), and immune selective anti-inflammatory derivatives (e.g., submandibular gland peptide-T and its derivatives).

Suitable anti-histamines include, but are not limited to, H1-receptor antagonists (e.g., acrivastine, azelastine, bilastine, brompheniramine, buclizine, bromodiphenhydramine, carbinoxamine, cetirizine, chlorpromazine, cyclizine, chlorpheniramine, clemastine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, meclizine, mirtazapine, olopatadine, orphenadrine, phenindamine, pheniramine, phenyltoloxamine, promethazine, pyrilamine, quetiapine, rupatadine, tripelennamine, and triprolidine), H2-receptor antagonists (e.g., cimetidine, famotidine, lafutidine, nizatidine, ranitidine, and roxatidine), tritoqualine, catechin, cromoglicate, nedocromil, and p2-adrenergic agonists.

Suitable anti-infectives include, but are not limited to, amebicides (e.g., nitazoxanide, paromomycin, metronidazole, tinidazole, chloroquine, miltefosine, amphotericin b, and iodoquinol), aminoglycosides (e.g., paromomycin, tobramycin, gentamicin, amikacin, kanamycin, and neomycin), anthelmintics (e.g., pyrantel, mebendazole, ivermectin, praziquantel, albendazole, thiabendazole, oxamniquine), antifungals (e.g., azole antifungals (e.g., itraconazole, fluconazole, parconazole, ketoconazole, clotrimazole, miconazole, and voriconazole), echinocandins (e.g., caspofungin, anidulafungin, and micafungin), griseofulvin, terbinafine, flucytosine, and polyenes (e.g., nystatin, and amphotericin b), antimalarial agents (e.g., pyrimethamine/sulfadoxine, artemether/lumefantrine, atovaquone/proguanil, quinine, hydroxychloroquine, mefloquine, chloroquine, doxycycline, pyrimethamine, and halofantrine), antituberculosis agents (e.g., aminosalicylates (e.g., aminosalicylic acid), isoniazid/rifampin, isoniazid/pyrazinamide/rifampin, bedaquiline, isoniazid, ethambutol, rifampin, rifabutin, rifapentine, capreomycin, and cycloserine), antivirals (e.g., amantadine, rimantadine, abacavir/lamivudine, emtricitabine/tenofovir, cobicistat/elvitegravir/emtricitabine/tenofovir, efavirenz/emtricitabine/tenofovir, abacavir/lamivudine/zidovudine, lamivudine/zidovudine, emtricitabine/tenofovir, emtricitabine/lopinavir/ritonavir/tenofovir, interferon alfa-2v/ribavirin, peginterferon alfa-2b, maraviroc, raltegravir, dolutegravir, enfuvirtide, foscarnet, fomivirsen, oseltamivir, zanamivir, nevirapine, efavirenz, etravirine, rilpivirine, delavirdine, nevirapine, entecavir, lamivudine, adefovir, sofosbuvir, didanosine, tenofovir, abacavir, zidovudine, stavudine, emtricitabine, zalcitabine, telbivudine, simeprevir, boceprevir, telaprevir, lopinavir/ritonavir, boceprevir, darunavir, ritonavir, tipranavir, atazanavir, nelfinavir, amprenavir, indinavir, saquinavir, ribavirin, valacyclovir, acyclovir, famciclovir, ganciclovir, and valganciclovir), carbapenems (e.g., doripenem, meropenem, ertapenem, and cilastatin/imipenem), cephalosporins (e.g., cefadroxil, cephradine, cefazolin, cephalexin, cefepime, cefazoline, loracarbef, cefotetan, cefuroxime, cefprozil, loracarbef, cefoxitin, cefaclor, ceftibuten, ceftriaxone, cefotaxime, cefpodoxime, cefdinir, cefixime, cefditoren, ceftizoxime, and ceftazidime), glycopeptide antibiotics (e.g., vancomycin, dalbavancin, oritavancin, and telavancin), glycylcyclines (e.g., tigecycline), leprostatics (e.g., clofazimine and thalidomide), lincomycin and derivatives thereof (e.g., clindamycin and lincomycin), macrolides and derivatives thereof (e.g., telithromycin, fidaxomicin, erythromycin, azithromycin, clarithromycin, dirithromycin, and troleandomycin), linezolid, sulfamethoxazole/trimethoprim, rifaximin, chloramphenicol, Fosfomycin, metronidazole, aztreonam, bacitracin, penicillin (amoxicillin, ampicillin, bacampicillin, carbenicillin, piperacillin, ticarcillin, amoxicillin/clavulanate, ampicillin/sulbactam, piperacillin/tazobactam, clavulanate/ticarcillin, penicillin, procaine penicillin, oxacillin, dicloxacillin, and nafcillin), quinolones (e.g., lomefloxacin, norfloxacin, ofloxacin, gatifloxacin, moxifloxacin, ciprofloxacin, levofloxacin, gemifloxacin, moxifloxacin, cinoxacin, nalidixic acid, enoxacin, grepafloxacin, gatifloxacin, trovafloxacin, and sparfloxacin), sulfonamides (e.g., sulfamethoxazole/trimethoprim, sulfasalazine, and sulfisoxazole), tetracyclines (e.g., doxycycline, demeclocycline, minocycline, doxycycline/salicylic acid, doxycycline/omega-3 polyunsaturated fatty acids, and tetracycline), and urinary anti-infectives (e.g., nitrofurantoin, methenamine, Fosfomycin, cinoxacin, nalidixic acid, trimethoprim, and methylene blue).

Suitable chemotherapeutics include, but are not limited to, paclitaxel, brentuximab vedotin, doxorubicin, 5-FU (fluorouracil), everolimus, pemetrexed, melphalan, pamidronate, anastrozole, exemestane, nelarabine, ofatumumab, bevacizumab, belinostat, tositumomab, carmustine, bleomycin, bosutinib, busulfan, alemtuzumab, irinotecan, vandetanib, bicalutamide, lomustine, daunorubicin, clofarabine, cabozantinib, dactinomycin, ramucirumab, cytarabine, Cytoxan, cyclophosphamide, decitabine, dexamethasone, docetaxel, hydroxyurea, dacarbazine, leuprolide, epirubicin, oxaliplatin, asparaginase, estramustine, cetuximab, vismodegib, asparaginase Erwinia chrysanthemi, amifostine, etoposide, flutamide, toremifene, fulvestrant, letrozole, degarelix, pralatrexate, methotrexate, floxuridine, obinutuzumab, gemcitabine, afatinib, imatinib mesylate, carmustine, eribulin, trastuzumab, altretamine, topotecan, ponatinib, idarubicin, ifosfamide, ibrutinib, axitinib, interferon alfa-2a, gefitinib, romidepsin, ixabepilone, ruxolitinib, cabazitaxel, ado-trastuzumab emtansine, carfilzomib, chlorambucil, sargramostim, cladribine, mitotane, vincristine, procarbazine, megestrol, trametinib, mesna, strontium-89 chloride, mechlorethamine, mitomycin, busulfan, gemtuzumab ozogamicin, vinorelbine, filgrastim, pegfilgrastim, sorafenib, nilutamide, pentostatin, tamoxifen, mitoxantrone, pegaspargase, denileukin diftitox, alitretinoin, carboplatin, pertuzumab, cisplatin, pomalidomide, prednisone, aldesleukin, mercaptopurine, zoledronic acid, lenalidomide, rituximab, octreotide, dasatinib, regorafenib, histrelin, sunitinib, siltuximab, omacetaxine, thioguanine (tioguanine), dabrafenib, erlotinib, bexarotene, temozolomide, thiotepa, thalidomide, Bacillus Calmette-Guerin (BCG), temsirolimus, bendamustine hydrochloride, triptorelin, arsenic trioxide, lapatinib, valrubicin, panitumumab, vinblastine, bortezomib, tretinoin, azacitidine, pazopanib, teniposide, leucovorin, crizotinib, capecitabine, enzalutamide, ipilimumab, goserelin, vorinostat, idelalisib, ceritinib, abiraterone, epothilone, tafluposide, azathioprine, doxifluridine, vindesine, and all-trans retinoic acid. KITS

Any of the compounds and/or formulations described herein (e.g., P188 and P188 containing formulations) can be presented as a combination kit. As used herein, the terms “combination kit” or “kit of parts” refers to the compounds, compositions, formulations, particles, cells and any additional components that are used to package, sell, market, deliver, and/or administer the combination of elements or a single element, such as the active ingredient, contained therein. Such additional components include, but are not limited to, packaging, syringes, blister packages, bottles, and the like. When one or more of the compounds, compositions, formulations, particles, cells, described herein or a combination thereof (e.g., agent(s)) contained in the kit are administered simultaneously, the combination kit can contain the active agent(s) in a single formulation, such as a pharmaceutical formulation, (e.g., a tablet, liquid preparation, dehydrated preparation, etc.) or in separate formulations. When the compounds, compositions, formulations, particles, and cells described herein or a combination thereof and/or kit components are not administered simultaneously, the combination kit can contain each agent or other component in separate pharmaceutical formulations. The separate kit components can be contained in a single package or in separate packages within the kit.

In some embodiments, the combination kit also includes instructions printed on or otherwise contained in a tangible medium of expression. The instructions can provide information regarding the content of the compounds and/or formulations, safety information regarding the content of the compounds and formulations (e.g., pharmaceutical formulations), information regarding the dosages, indications for use, and/or recommended treatment regimen(s) for the compound(s) and/or pharmaceutical formulations contained therein. In some embodiments, the instructions can provide directions and protocols for administering the compounds and/or formulations described herein to a subject in need thereof. In some embodiments, the instructions can provide one or more embodiments of the methods for administration of the P188 and/or pharmaceutical formulation thereof such as any of the methods described in greater detail elsewhere herein.

### Methods of Preventing Non-Irradiated Cell Damage During Radiation Exposure

Described in several exemplary embodiments herein are methods of preventing non-insulted cell damage during and/or post-insult. As previously discussed, the insult can be any insult, such as energetic insult (e.g., radiation), chemical insult, biological insult (e.g., viral or microorganism), physiological insult (e.g., ischemic event, such as myocardial infarction or stroke), and/or mechanical insult (e.g., heat, cold, pressure, lysis, etc.). Exposure to the insult can be intentional, such as for therapy, or can be accidental, such as being unintentionally exposed to the insult in the environment. The insult can be unplanned or unexpected, such as in the case of a physiological insult.

In general, the method can include administering one or more amounts of P188 or a pharmaceutical formulation thereof before, during, and/or after in insult. The one or more amounts of P188 or pharmaceutical formulation thereof can be administered for 0-48 prior to and/or 0-48 hours after insult. In some embodiments, it is preferred to administer the one or more amounts of P188 prior to exposure to an insult. It will be appreciated that in some embodiments, administration of P188 post insult may not provide as much of a benefit as compared with P188 administration prior to the insult, but nonetheless a benefit can be achieved. It will also be appreciated that, particularly in cases where exposure to an insult cannot be predicted or planned (e.g., in the case of an ischemic event or accidental radiation exposure), that it is advantageous to administer one or more amounts of P188 post insult relative to no administration at all. In some embodiments, the one or more amounts of P188 given post-insult are given as close to the time of insult as possible. For example, during an ischemic event this may be prior to catherization and tpa.

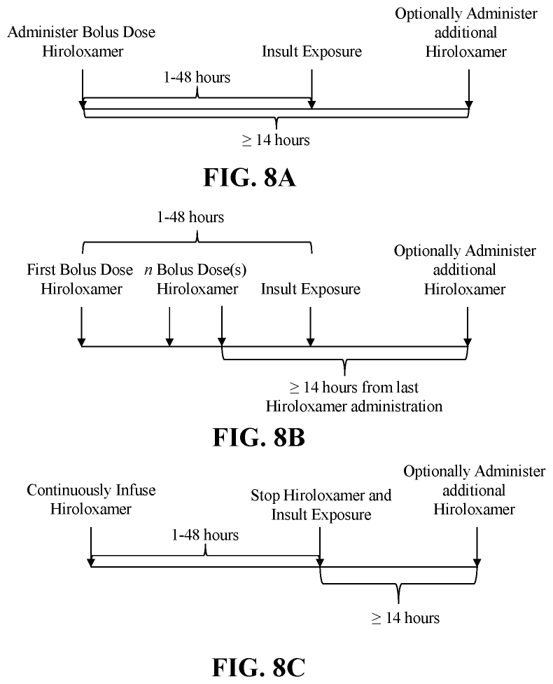

In some exemplary embodiments, the insult is radiation and thus, described herein are methods of preventing non-irradiated cell damage during radiation, such as during radiation therapy by way of example only. FIGS. 8A-8C show embodiments of the methods, which are further discussed in greater detail below.

In some embodiments, the method can include administering a first amount of P188 or a pharmaceutical formulation thereof to a subject prior to exposure of one or more regions of the subject to an insult; and exposing one or more regions of the subject to an insult after the blood concentration of P188 reaches 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). As shown in FIG. 8A, the first amount can be a single bolus dose, such that at a particular point in time after administration the blood concentration of the P188 will reach a concentration between 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL), at which point one or more regions of the subject can be exposed to the insult. In some embodiments, the area exposed to the insult contains one or more cancerous cells. In some embodiments and shown in FIG. 8A, the subject does not receive any further amount of P188 or a pharmaceutical formulation thereof for at least about 14-24 hours after receiving the first amount of P188 or a pharmaceutical formulation thereof.

In some embodiments, the first amount is such that when given as a single bolus dose the blood concentration of P188 reaches a concentration between 1 and about 5 mg/mL (e.g., about 1, 1.1, 1.2, L3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL) within about 1 to about 48 hours. In some embodiments, the first amount is such that when given as a single bolus does the blood concentration of P188 reaches a concentration between about 1 and about 5 mg/mL (e.g., about 0, 0.25, 0.5, 0.75, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL) within about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose of P188 or a pharmaceutical formulation thereof. Thus, in some embodiments one or more regions of the subject can be exposed to ionizing radiation about 0 hours (i.e., at the same time as insult)-48 hours after administration of the first amount of P188 or a pharmaceutical formulation thereof. In some embodiments, one or more regions of the subject can be exposed to ionizing radiation about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose of P188 or a pharmaceutical formulation thereof.

In some embodiments, a subject's blood concentration of P188 or a pharmaceutical formulation thereof can be measured at one or more time points after administration of the first amount to determine the time point at which to expose one or more regions of the subject to radiation. In some embodiments, the time for exposing one or more regions of the subject to radiation can be calculated based on e.g., the amount of P188 administered, the half-life of P188 (which is about 4.5 hours), and subject characteristics (e.g., age, weight, medical condition, and others that will be appreciated by a medical practitioner).

As shown in FIG. 8B, multiple bolus doses of P188 or a pharmaceutical formulation thereof can be administered. The total number of additional doses are designated elsewhere herein and in FIG. 8B as “n”. Any particular dose after the first amount given as a bolus dose can be referred to as the “n^{th}” dose. For example, dose 5 given out of a total of 8 can be referred to as the 5^{th} dose. In some contexts, the n^{th} dose can be referring to the last dose administered. In some embodiments, the number of doses that can be administered after the first dose can be 2, 3, 4, 5, 6, 7, 8, 9, 10, 11, 12, 13, 14, 15, 16, 17, 18, 19, or 20. In some embodiments, the amount of any of the doses can be less than what would achieve a P188 blood concentration of between 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL) if given as a single dose, but cumulatively cause the P188 blood concentration to reach between 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). Similar to that discussed with respect to the embodiments exemplified in FIG. 8A, the amounts needed in each bolus dose administered can be determined by measuring P188 concentration in the blood stream or can be calculated based on e.g., the amount of P188 administered, the half-life of P188 (which is about 4.5 hours), and subject characteristics (e.g., age, weight, medical condition, and others that will be appreciated by a medical practitioner).

Like in the embodiments exemplified and discussed with respect to FIG. 8A, the total amount of P188 administered across all the bolus doses the blood concentration of P188 reaches a concentration between 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL) within about 1 to about 48 hours, such as within about within 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours of the first, n^{th}, or last dose administered. Thus, in some embodiments, one or more regions of the subject can be exposed to an insult about 0 hours (i.e., at the same time as insult exposure)-48 hours after administration of the first amount, n^{th} amount, or last amount of P188 or a pharmaceutical formulation thereof. In some embodiments, one or more regions of the subject can be exposed to an insult about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose, n^{th} dose, or last dose of P188 or a pharmaceutical formulation thereof.

In some embodiments, the subject does not receive further P188 for at least 14-24 hours after the last bolus dose of P188 or pharmaceutical formulation thereof is administered.

As exemplified in FIG. 8C, the P188 or pharmaceutical formulation thereof can be continuously infused into a subject for a period of time prior to exposure of one or more regions of the subject to an insult. In some embodiments, the period of time can be about 0-48 hours, such as about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours. In some embodiments, the P188 or pharmaceutical formulation thereof can be continuously infused into a subject until the blood concertation of P188 reaches a concentration between about 1 and about 5 mg/mL (e.g., about 1, 1.1, 1.2, L3, 1.4, 1.5, 1.6, 1.7, 1.8, 1.9, 2, 2.1, 2.2, 2.3, 2.4.2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). In some embodiments, the P188 blood concentration reaches this amount within about 1 to about 48 hours, such as in about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours.

In some embodiments, a method of preventing damage to non-insulted cells in a subject during and/or after insult exposure can include administering an amount of poloxamer 188 (P188) or a pharmaceutical formulation thereof to a subject prior to exposure of one or more regions of the subject to an insult, wherein the amount ranges from about 10 to about 150 mg/kg; and exposing one or more regions of the subject to ionizing radiation (or other insult) within 0-48 hours after administering the amount of P188 or pharmaceutical formulation thereof. In some embodiments, the amount can range from about 100-150 mg/kg. In some embodiments the amount can range from about 20-40 mg/kg. In some embodiments, a first dose (e.g., a loading dose) can range from about 100 to about 150 mg/kg. In some embodiments a second amount can be delivered one or more times for maintenance and can range from about 20 to about 40 mg/kg. In some embodiments, the maintenance dose can be administered one or more times per hour. In some embodiments, exposing one or more regions of the subject to an insult can occur within about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the amount of the P188 or formulation thereof.

In some embodiments, the insulted cells can be diseased, abnormal, or otherwise unhealthy cells. In some embodiments, the insulted cells can be cancerous. In some embodiments the insulted cells can be non-cancerous tumor or non-tumor cells. The non-insulted cells can be normal or otherwise healthy cells. In some embodiments, the non-insulted cells are soft-tissue cells. In some embodiments, the non-insulted cells are vascular cells. In some embodiments, the non-insulted cells are endothelial cells.

Also described herein are methods of treating a disease or condition. In some embodiments, the disease or condition can be caused by an insult to a cell or tissue. In some embodiments, the disease or condition can be a disease or condition that causes production of free radicals or oxidation in/by affected cells and/or stimulates an inflammatory response or cascade. In some embodiments, the treatment for a disease or condition can cause an insult to a cell or tissue. In some embodiments, the disease or condition can be a virus. In some embodiments, the disease or condition can be acute respiratory distress syndrome (ARDS). In some embodiments, disease or condition can be a cardiac condition. In some embodiments, the disease or condition can be a neurological disease or condition. In some embodiments the disease or condition is acute radiation sickness (ARS) (see e.g., Williams and McBride, W. H. 2011. Int Radiat Biol 87(8):851-868. In some embodiments, the disease or disorder is a radiation toxicity. In some embodiments, the disease or disorder is an insult, such as radiation-induced pneumonitis. In some embodiments, the disease or disorder is an ischemic event (such as stroke or myocardial infarction).

In some embodiments, the method of treating a disease or condition can include administering a first amount of poloxamer 188 (P188) or a pharmaceutical formulation thereof to a subject prior to exposure of one or more regions of the subject to an insult; and exposing one or more regions of the subject to an insult after the blood concentration of P188 reaches between about 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). In some embodiments, a method a disease or condition can include administering a first amount of poloxamer 188 (P188) or a pharmaceutical formulation thereof to a subject prior to exposure of one or more regions of the subject to an insult; and exposing one or more regions of the subject to an insult 0 hours (i.e., at the time of insult) to 48 hours after administering the first amount of P188 or a pharmaceutical formulation thereof, such as 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose of P188 or a pharmaceutical formulation thereof. In some embodiments, the method can include administering multiple doses of P188 or pharmaceutical formulation thereof, similar to that described above with respect to FIG. 8B. In some embodiments, the method can include continuous administration of P188 or a pharmaceutical formulation thereof, in similar fashion to that described above with respect to FIG. 8C.

Also described herein are methods of treating a non-cancerous tumor or condition. In some embodiments, a method of treating a non-cancerous tumor or condition can include administering a first amount of poloxamer 188 (P188) or a pharmaceutical formulation thereof to a subject prior to exposure of one or more regions of the subject to ionizing radiation; and exposing one or more regions of the subject to ionizing radiation after the blood concentration of P188 reaches between about 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL) In some embodiments, a method of treating a non-cancerous tumor or condition can include administering a first amount of poloxamer 188 (P188) or a pharmaceutical formulation thereof to a subject prior to exposure of one or more regions of the subject to ionizing radiation; and exposing one or more regions of the subject to ionizing radiation 0-48 hours after administering the first amount of P188 or a pharmaceutical formulation thereof, such as about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose of P188 or a pharmaceutical formulation thereof. In some embodiments, the method can include administering multiple doses of P188 or pharmaceutical formulation thereof, similar to that described above with respect to FIG. 8B. In some embodiments, the method can include continuous administration of P188 or a pharmaceutical formulation thereof, in similar fashion to that described above with respect to FIG. 8C.

In some embodiments, the non-cancerous tumor (i.e., a benign tumor) or other non-cancerous condition can be an acoustic neuroma, arteriovenous malformation, the thyroid, trigeminal neuralgia, meningioma, an inflammatory/proliferative disorder (e.g., Dupuytren's disease, heterotopic ossification, keloid scarring, pigmented villonodular synovitis), other benign tumors, and other conditions (see also e.g., McKeown et al., (Br J. Radiol. 2015 88(1056), particularly at Table 1; Seegenschmiedt et al. Br J Radiol. 2015: 88(1051)).

Also described herein are methods of treating a cancer. In some embodiments, a method of treating a cancer can include administering a first amount of poloxamer 188 (P188) or a pharmaceutical formulation thereof to a subject prior to exposure of one or more regions of the subject to ionizing radiation; and exposing one or more regions of the subject to ionizing radiation after the blood concentration of P188 reaches between 1 and about 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). In some embodiments, a method of treating a cancer can include administering a first amount of poloxamer 188 (P188) or a pharmaceutical formulation thereof to a subject prior to exposure of one or more regions of the subject to ionizing radiation; and exposing one or more regions of the subject to ionizing radiation 0-48 hours after administering the first amount of P188 or a pharmaceutical formulation thereof, such as about 0 (i.e., delivery at time of insult) 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours after administering the first dose of P188 or a pharmaceutical formulation thereof. In some embodiments, the method can include administering multiple doses of P188 or pharmaceutical formulation thereof, similar to that described above with respect to FIG. 8B. In some embodiments, the method can include continuous administration of P188 or a pharmaceutical formulation thereof, in similar fashion to that described above with respect to FIG. 8C.

In some embodiments, the cancer is a solid tumor cancer. In some embodiments, the cancer is a non-solid tumor. In some embodiments, the cancer is a spinal cord tumor, spine tumor, a leukemia, a head or neck cancer, Ewing's sarcoma, a soft tissue sarcoma, prostate cancer, an esophageal cancer, colorectal cancer, paranasal sinus cancer, pancreatic cancer, chordoma, osteosarcoma, chondrosarcoma, breast cancer, meningioma, a brain tumor, a bone cancer, a lung cancer, a lymphoma (including but not limited to non-Hodgkin's lymphoma), a liver cancer, or a combination thereof.

In some embodiments, the amount of ionizing radiation that one or more regions of the subject are exposed to is about 100 Gy or less, but greater than zero. In some embodiments, the amount of ionizing radiation that one or more regions of the subject are exposed ranges from about 1, to about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Gy. In some embodiments, the amount of ionizing radiation that one or more regions of the subject are exposed to is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Gy.

In some embodiments, the amount of ionizing radiation that one or more regions of the subject are exposed to is about 80 Gy or less, but greater than zero. In some embodiments, the amount of ionizing radiation that one or more regions of the subject are exposed ranges from about 1, to about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 Gy. In some embodiments, the amount of ionizing radiation that one or more regions of the subject are exposed to is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 Gy.

In some embodiments, the amount of ionizing radiation that one or more regions of the subject are exposed to is 50 Gy or less, but greater than zero. In some embodiments, the amount of ionizing radiation that one or more regions of the subject are exposed ranges from about 3 to about 50 Gy, such as about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 Gy. In some embodiments, the amount of ionizing radiation that one or more regions of the subject are exposed to can be 50 Gy or more. In some embodiments, the amount of ionizing radiation that one or more regions of the subject are exposed to can be about 50 to about 100 Gy, such as about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 Gy. In some embodiments the exposure to ionizing radiation is accidental. In some embodiments, the subject has developed or is at risk to develop acute radiation syndrome. In some embodiments, the subject can be exposed to >0.05 Gy (e.g., 0.5, 0.6, 0.7, 0.8, 0.9, 1.0, 2.0, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 Gy).

Also described herein are methods of preventing insult-induced pneumonitis in a subject. In some embodiments, the method includes a) administering an amount of P188 or a formulation thereof to a subject prior to exposure to an insult; b) administering an amount of P188 or a formulation thereof to a subject immediately following exposure to an insult; c) administering an amount of P188 or a formulation thereof to a subject during an insult; or d) a combination thereof. In some embodiments, the amount is effective to increase the blood concentration of P188 in the subject to between 1 mg/mL and 5 mg/mL (e.g., about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or about 5 mg/mL). In some embodiments, the amount is effective to coat one or more non-insulted cells. In some embodiments, the amount is effective to reduce or prevent oxidative damage to one or more non-insulted cells. In some embodiments, the amount is effective to reduce or prevent inflammatory damage to one or more non-insulted cells. In some embodiments, administering occurs 0-48 hours prior to exposure to the insult, such as about 0 (i.e., administering at time of insult) 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, 24, 24.25, 24.5, 24.75, 25, 25.25, 25.5, 25.75, 26, 26.25, 26.5, 26.75, 27, 27.25, 27.5, 27.75, 28, 28.25, 28.5, 28.75, 29, 29.25, 29.5, 29.75, 30, 30.25, 30.5, 30.75, 31, 31.25, 31.5, 31.75, 32, 32.25, 32.5, 32.75, 33, 33.25, 33.5, 33.75, 34, 34.25, 34.5, 34.75, 35, 35.25, 35.5, 35.75, 36, 36.25, 36.5, 36.75, 37, 37.25, 37.5, 37.75, 38, 38.25, 38.5, 38.75, 39, 39.25, 39.5, 39.75, 40, 40.25, 40.5, 40.75, 41, 41.25, 41.5, 41.75, 42, 42.25, 42.5, 42.75, 43, 43.25, 43.5, 43.75, 44, 44.25, 44.5, 44.75, 45, 45.25, 45.5, 45.75, 46, 46.25, 46.5, 46.75, 47, 47.25, 47.5, 47.75, or about 48 hours prior to insult. In some embodiments, administering occurs 0 (i.e., administering at time of insult)-24 hours post exposure to the insult, such as about 0, 0.25, 0.5, 0.75, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5.6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 20.25, 20.5, 20.75, 21, 21.25, 21.5, 21.75, 22, 22.25, 22.5, 22.75, 23, 23.25, 23.5, 23.75, or about 24 hours post-insult. In some embodiments, the insult is a mechanical insult, a chemical insult, a biological insult, an energetic insult, or a combination thereof. In some embodiments, the insult is ionizing radiation.

In some embodiments, the amount of ionizing radiation is about 100 Gy or less, but greater than zero. In some embodiments, the amount of ionizing radiation that one or more regions of the subject are exposed ranges from about 1 to about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Gy. In some embodiments, the amount of ionizing is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 Gy.

In some embodiments, the amount of ionizing radiation is about 80 Gy or less, but greater than zero. In some embodiments, the amount of ionizing radiation ranges from about 1 to about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80 Gy. In some embodiments, the amount of ionizing radiation is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 Gy.

In some embodiments, the amount of ionizing radiation is 50 Gy or less. In some embodiments, the amount of ionizing radiation can range from about 3 to about 50 Gy, such as about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 Gy. In some embodiments, the amount of ionizing radiation can be 50 Gy or more. In some embodiments, the amount of ionizing radiation can be about 50 to about 100 Gy, such as about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 Gy. In some embodiments exposure to ionizing radiation is accidental. In some embodiments, the subject has developed or is at risk to develop acute radiation syndrome. In some embodiments, the subject can be exposed to >0.05 Gy (e.g., 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 Gy).

In some embodiments, in addition to pretreating with the P188 formulations as described above, the method can also include administering an amount of a P188 formulation described herein immediately (e.g., within 1-24 hours and/or when the insult is still within the early phases and proximate/remote cell damage is still mediated by oxidation) post insult.

In some embodiments, particularly when exposure to an insult cannot be predicted and thus pretreating with P188 or a pharmaceutical formulation thereof is unavailable, one or more amounts of P188 or a pharmaceutical formulation thereof is/are administered one or more times 0-48 hours post insult, such as about 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or about 48 hours post insult. Exemplary unpredictable insult exposure where concurrent or post-insult administration P188 is the only option are e.g., a physiological event (e.g., an ischemic event) and accidental radiation exposure. Others will be appreciated in view of the disclosure herein.

In some embodiments, the method can include administering a co-therapy, such as an auxiliary active agent that can be administered as a stand-alone pharmaceutical formulation or be included in the poloxamer formulation. Where co-therapies, auxiliary agents, and/or multiple pharmaceutical formulations are to be delivered to a subject and/or a cell, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time. In some embodiments, the auxiliary agent, co-therapy, and the like can be administered before, after or both before and after the poloxamer formulation. In some embodiments, the methods described herein are a co-therapy to treating an insult, such as a biological insult. In some embodiments, the methods described herein are a co-therapy where the primary treatment causes an insult to cells, such as diseased cells.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1—Hiroloxamer Protects Cells Against Hydrogen Peroxide

During radiation therapy for the treatment of cancer, the vasculature unavoidably gets exposed to radiation. Ionizing radiation causes cell death either directly by damaging cell's DNA, or indirectly through the production of noxious by-products, such as free radicals, reactive oxygen species, etc. The vasculature responds to ionizing radiation in two phases. The acute/early phase occurs within 24 hours, and directly damaged cells undergo cell death, which compromises the barrier integrity of the vasculature. Cells indirectly exposed to the by-products send out damage signals and induce a pro-inflammatory response. If these complications do not resolve, the late effects begin to manifest, including capillary collapse and scar formation.

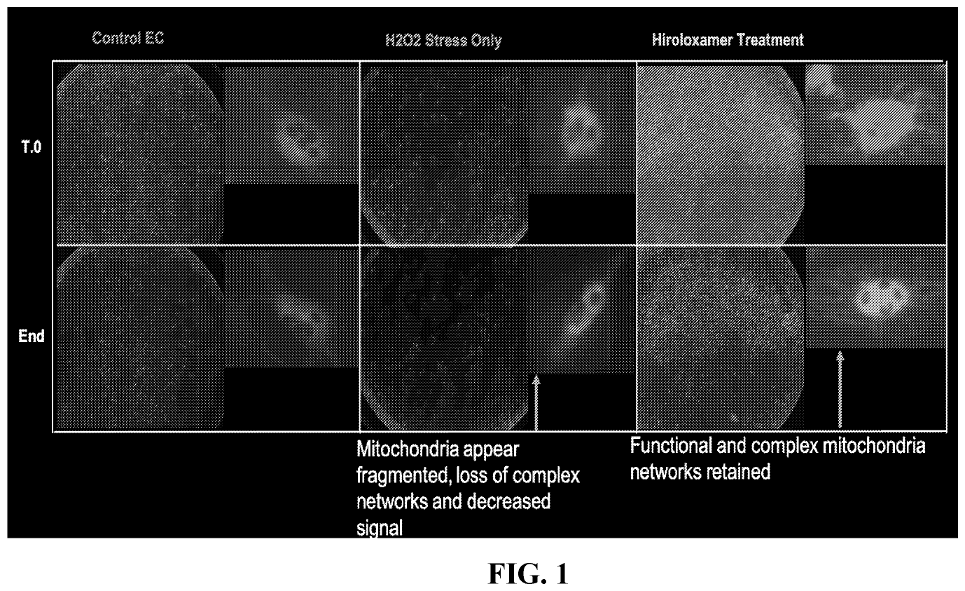

As can at least be demonstrated by FIG. 1, Hiroloxamer was observed to provide protection to human adipose microvascular endothelial cells when exposed to hydrogen peroxide and prevented damage to these cells. Briefly, human adipose microvascular endothelial cells (HAMEC) were stained with MitoTracker Deep Red FM to label functional mitochondria and seeded into 12-well plates. The cells were allowed to adhere and grow to confluence. Once confluent, appropriate wells received a three-hour treatment of Hiroloxamer. After three hours of incubation, 1 mM hydrogen peroxide was added to each well overnight. The ability of Hiroloxamer to provide protection to endothelial cells exposed to hydrogen peroxide was evidenced by the presence of complex mitochondrial networks (FIG. 1, greyscale) observed in the Hiroloxamer treatment group, but not in the untreated group.

### Example 2—Hiroloxamer does not Interfere with Radiation Effect on Cancer Cells

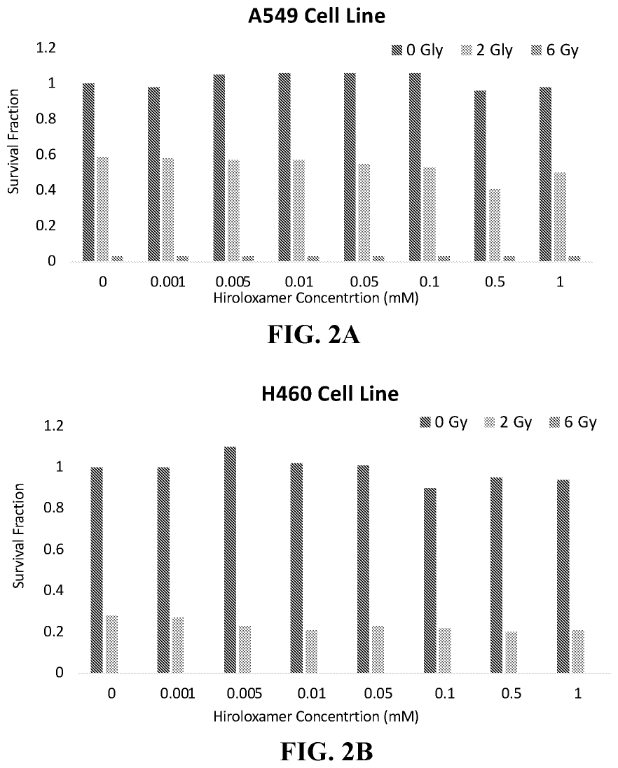

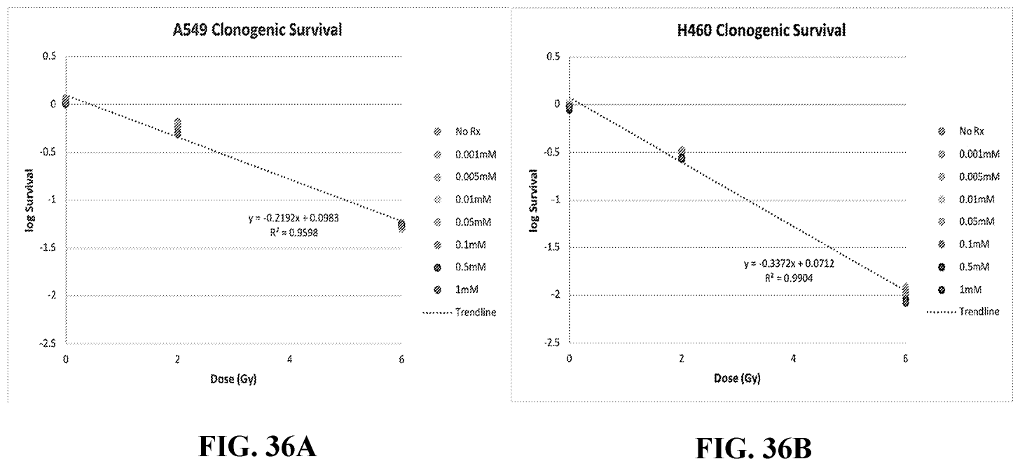

In the context of radiotherapy, it is important that the Hiroloxamer treatment does not interfere with the capability of irradiation to effectively kill cancer cells. Here it can be demonstrated that Hiroloxamer treatment prior to radiation therapy does not interfere with the effect irradiation on cancer cells (FIGS. 2A-2B and 36A-36B). Two cancer cell lines, H460 and A549, were used to verify that Hiroloxamer treatment prior to radiation therapy will not interfere with the capability of RT to effectively kill cancer cells. Briefly, cells were cultured and exposed to the indicated concentrations of Hiroloxamer for 3 hours prior to exposure to 0, 2, or 6 Gy of ionizing radiation. Then the survival fraction was determined for each group. There were no significant differences between control and treatment and any concentration of Hiroloxamer.

### Example 3—Effect of Hiroloxamer on Healthy Microvascular Endothelial Cells

Following the results demonstrated in Example 2 indicating Hiroloxamer administration does not impact the efficacy of radiation therapy to kill cancer cells, its effects on healthy human adipose microvascular endothelial cells (HAMEC) were investigated. Cells were seeded into wells of a 12-well plate and incubated for two days prior to irradiation. To investigate the effects of Hiroloxamer as a prophylactic and mitigative agent, 0.2 mM Hiroloxamer in endothelial growth medium-2 (EGM2, Lonza) was administered at different time points: about 3 hours prior to irradiation (Rx pretreatment), about 30 minutes following irradiation (Rx@0.5), and about 24 hours after irradiation (Rx@24). The 12-well plates were individually placed in a X-Rad320 and directly exposed to 0.5, 1, 2, 4, 10, 20, or 50 Gy of X-ray irradiation. Following irradiation, the Rx@0.5 group was supplemented with the appropriate volume of drug for the dose and allowed to incubate overnight. The following day, all medias were changed, and refreshed with EGM-2. The Rx@24 Hiroloxamer administration was performed at this time, and the Hiroloxamer was removed the following day. The Hiroloxamer was administered for 24 hr for each group, and then removed and replaced with fresh EGM-2. Live cell imaging was performed on a LionHeartFX throughout the duration of the study (every 24 hr). The plates were allowed to recover to confluence and fixed in 2% PFA for analysis. The wells were stained with Phalloidin 633 (F-Actin) and Hoescht for nuclei.

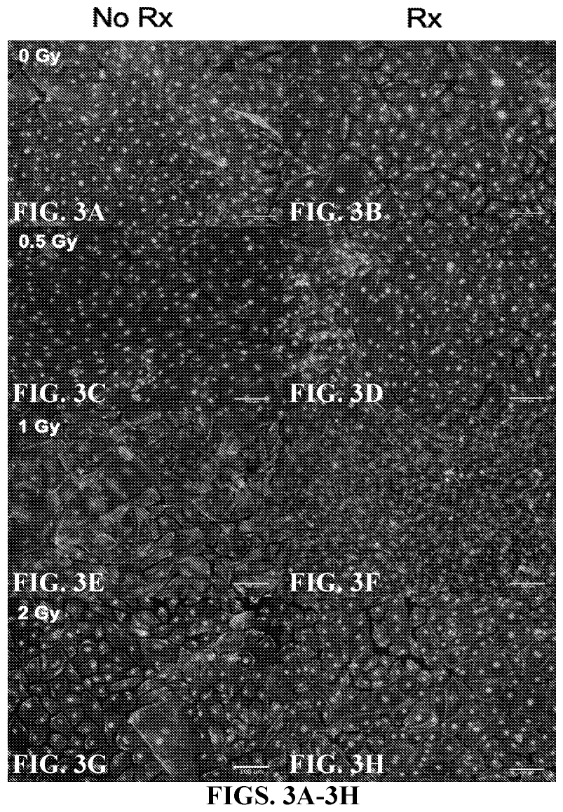

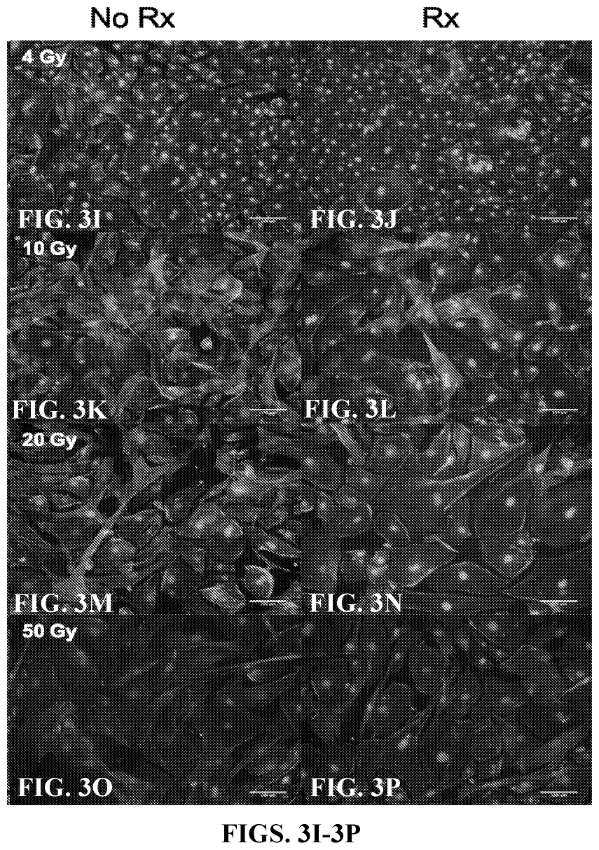

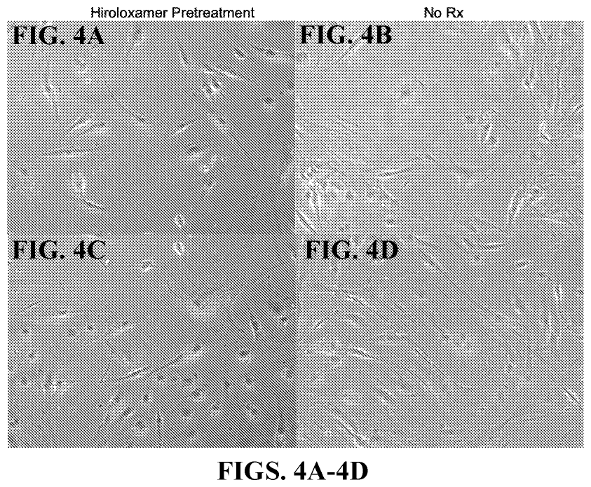

Like how endothelial cells organize in the vasculature, in a dish they make tight cell-cell adhesions to form a compact monolayer across the dish. The cell has an internal cytoskeleton, composed mainly of F-actin, that regulates cell shape, provides strength, and is highly dynamic depending on cellular demand. In confluent monolayers the cells internal cytoskeleton architecture is oriented in a unilateral direction, anisotropic homogeneity. In these monolayers and in the vasculature, the endothelial cells are directly linked via adhesion complexes (adherens junctions) that directly connect with the internal cytoskeleton. These complexes provide a barrier that regulate vascular permeability. The distinct green outline of each individual cell in FIGS. 3A-3P, Panel Control is the cortical actin rim, which determines cell shape. It is associated with cell-cell and cell-matrix adhesion complexes that keep cells tightly adhered to each other and to the matrix (e.g., for maintenance of endothelial barrier integrity that regulates vascular permeability). Ionizing radiation is known to damage the endothelium and disrupt their barrier function through the disruption of cell-cell adhesions and the actin cytoskeleton. Human adipose microvascular endothelial cells directly exposed to 0.5 Gy X-ray irradiation recovered to confluent monolayers with typical F-actin expression with or without drug treatment (FIGS. 3A-3B, Panel 0.5Gy). Cells have been reported to recover into confluent monolayers are this dose.

Moderate doses of irradiation (1-4 Gy) induce actin reorganization from its cortical distribution into stress fibers that span throughout the cell body. This remodeling of the cells' cytoskeleton leads to cell contraction and the loss of junctions between adjacent cells, leading to increased vascular permeability in vivo. This effect was observed in the cells exposed to irradiation without Hiroloxamer pretreatment (FIGS. 3A-3B, Panels 1,2,4Gy). In the Hiroloxamer pretreatment group, however, the cells re-established the cortical actin rim and formed tight cell-cell junctions with no intercellular gaps evident. Higher doses of ionizing radiation (about 10-50 Gy) lead to an increase in cell surface area in groups, likely due to the loss of cell-cell adhesions and secondary damage from indirect effects of radiation (FIGS. 3A-3B, Panels 10, 20, 50Gy). The 10 Gy Hiroloxamer group notably had more cells that appeared not to have increased in cell surface area, look activated and migratory, potentially trying to re-establish cell-cell adhesions that were lost upon exposure (FIGS. 3A-3B, Panel 10Gy). At the higher doses, 20 and 50 Gy, there was evident increase in cell surface area in all groups, however, the Hiroloxamer pretreatment appeared to prevent blebbing and explosion of cell membranes, where the cells appear rounded and intact compared to the non-treated group (FIGS. 3A-3B, Panels 20, 50Gy).

### Example 4—Protection of Non-Irradiated Cells Adjacent to Irradiated Cells

This Example can demonstrate at least that Hiroloxamer can protect human adipose microvascular endothelial cells directly adjacent to areas exposed to ionizing radiation. The endothelial cells were stained with MitoTracker Deep Red to label the mitochondria for live cell imaging. HAMECs were seeded into 60 mm Petri dishes and allowed to grow until confluent. Three hours prior to irradiation, Hiroloxamer was added to the appropriate dishes. Each Petri dish was placed in the irradiator with a lead shield placed on top having a 6 mm hole for radiation exposure to pass through and expose the cells to 10 Gy irradiation within this area (10% of the total plate area). Following irradiation, the plates were returned to the incubator overnight. Media was changed with normal growth medium the following day, and all plates were fixed 48 hours after irradiation. Different areas were assessed for mitochondrial potential following irradiation: within the irradiated/exposed (6 mm) area, directly next to the exposed area, and far away from the exposed area. The intensity of MitoTracker Deep Red (MTDR) changes with mitochondrial potential, i.e. viable cells appear as highly fluorescent population, whereas apoptotic cells exhibit a lower fluorescence. The intensity and structure of the mitochondria exhibited with the MitoTracker Deep Red dyes were compared with corresponding phase contrast images to check the integrity of the cell membrane. Cells with low MTDR signal and disrupted cell membrane were considered dead.

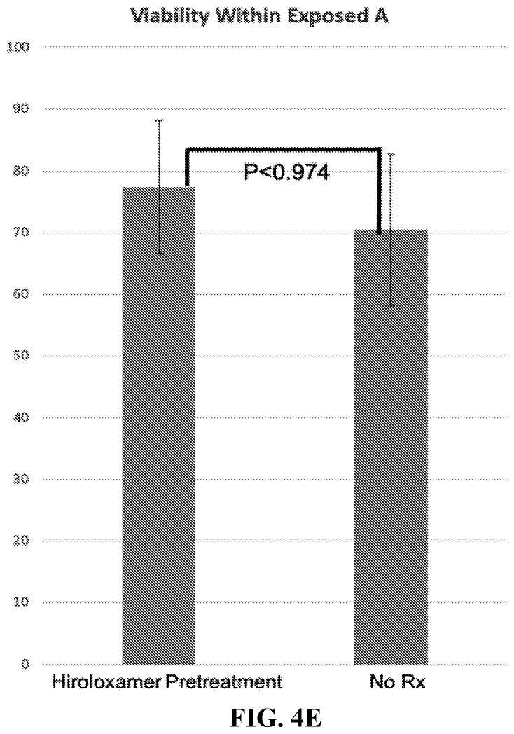

Within the irradiated area, i.e. within the area that irradiation was able to pass through the 6 mm shield, there was no difference in cell viability between the different groups (FIGS. 4A-4E; n=16; p<0.974). This was an expected result, as the Hiroloxamer treatment had no effect on the directly exposed cancer cells.

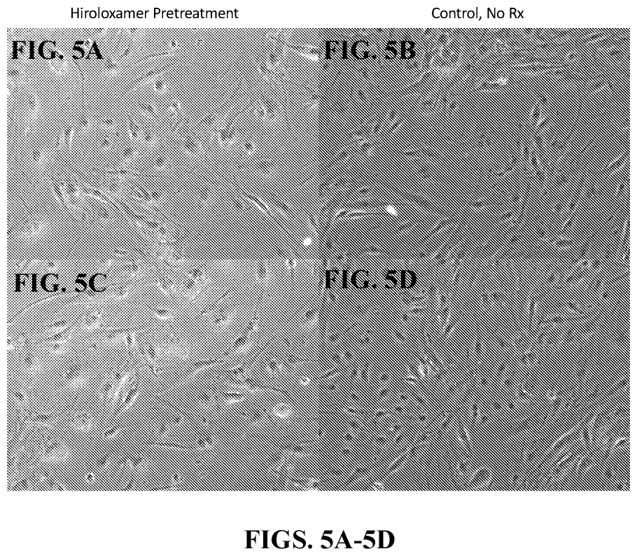

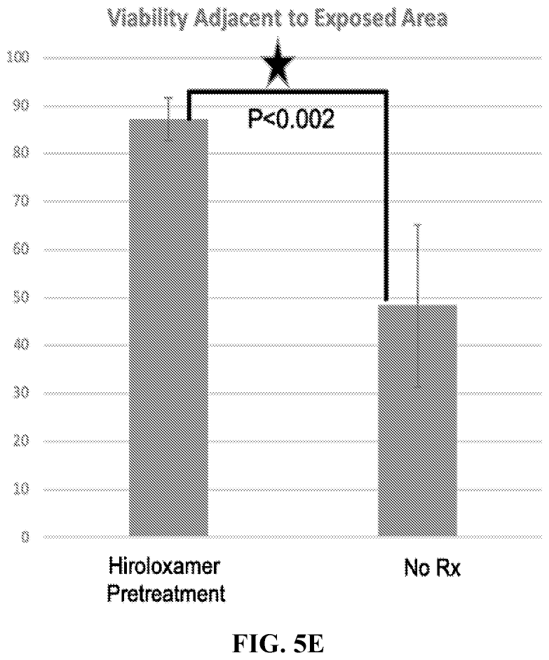

Areas directly adjacent to the irradiated area, but outside of the 6 mm exposed zone, was also evaluated for viability. In the regions proximal to the irradiated area, there was a significant increase in the number of MitoTracker Deep Red-positive cells with intact cell membranes, validated by corresponding phase contrast images, in the Hiroloxamer treatment group as compared to the untreated control group (n=9; p<0.002; FIGS. 5A-5E).

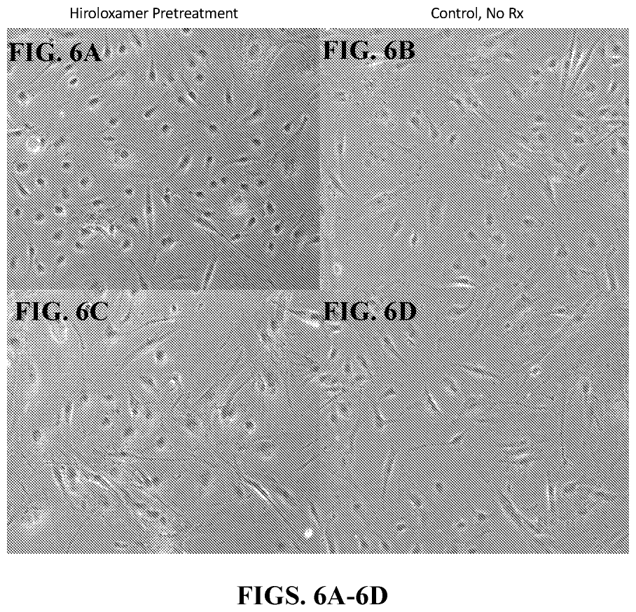

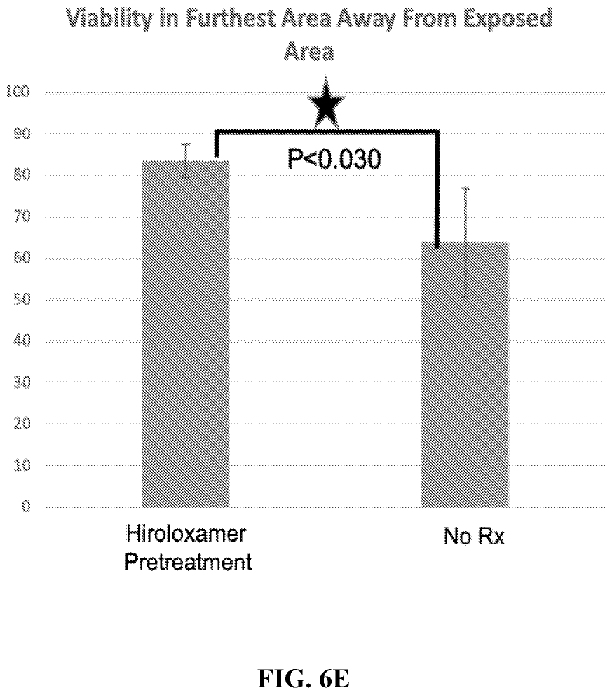

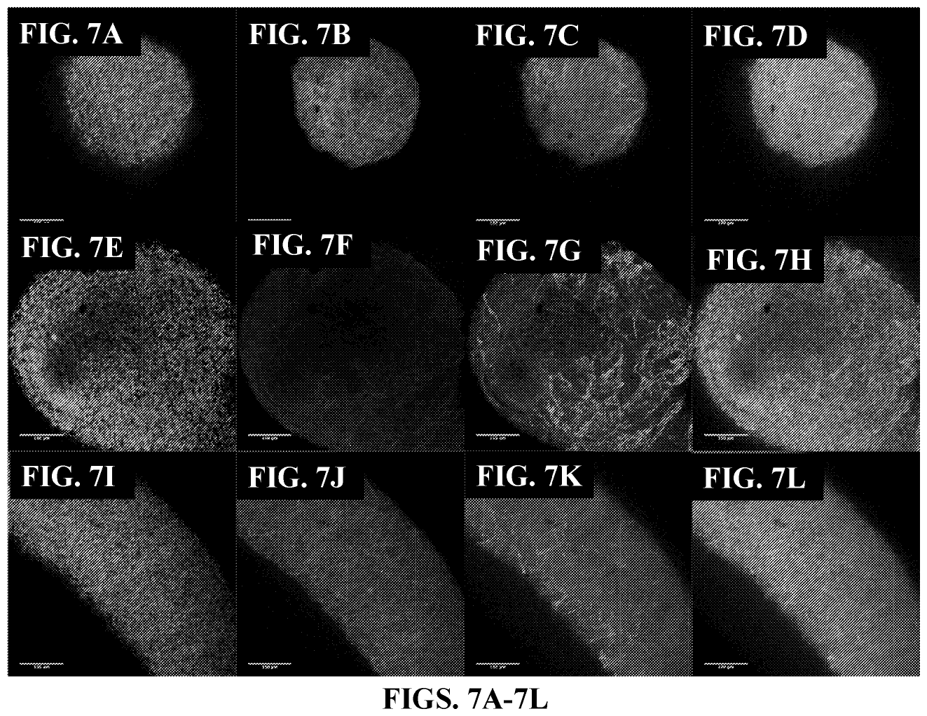

Finally, areas furthest away from the irradiated area of the dish were compared (FIGS. 6A-6E), There was a significant increase in MitoTracker Deep Red-positive cells in the Hiroloxamer treatment group as compared to the control group (n=4, p<0.030; FIGS. 5A-5E). The number of samples in this group was smaller due to the limited size of the dish and maintaining a distance of at least 30 mm from the irradiated area.

### Example 5—Effects of Pre-Irradiation Administration of Hiroloxamer on 3D Tissue Constructs

The effects of Hiroloxamer administration prior to radiation exposure on three-dimensional tissue engineered constructs was investigated. There are previous reports on these constructs in the past, Pattanaik, S., et. al. 2018. The Scaffold-free Prevascularized Endothelial-fibroblast Constructs (SPECs) are formed by seeding Normal Human Dermal Fibroblasts and Human Adipose Microvascular Endothelial Cells (4:1) in a non-adherent agarose mold, and culturing for 4 days in normal medium (FGM-2:EGM-2, 2:1). The SPECs were seeded, and on day 3 Hiroloxamer administration was given three hours prior to exposure to radiation. The constructs were exposed to 10Gy X-ray irradiation in the X-Rad320 irradiator set to 320.0 kV and 12.50 mA. The constructs were returned to the incubator overnight and fixed the following day with 4% PFA. The constructs were then stained with Phalloidin 633, Hoescht, and CD31 for analyses of the actin cytoskeleton and endothelial networks within the construct. All constructs were imaged on the Leica SP5 confocal microscopy with the same channel settings, and similar stack sizes.

FIGS. 7I-7L show Rx pretreatment, exposed to 10 Gy (88.031 μm with 4 μm step size). In control SPEC (No Rx, No XRT) there are lacy endothelial networks (red) that span across the construct embedded in an abundant and consistent F-actin network (green) throughout the entirety of the construct (FIGS. 7A-7D). The formed endothelial networks resemble primitive capillary-like networks in vivo (Pattanaik). When the SPEC was exposed to 10Gy of X-ray irradiation, there was evident actin depolymerization (indicated by the decrease in F-actin expression), and the endothelial networks became enlarged and disorganized (FIGS. 7E-7H). The changes in the vascular component of the construct are similar to the changes seen in the vasculature in vivo in response to ionizing radiation, where there is a loss in cell-cell adhesions leading to increased vascular permeability. The increased size of the vascular networks within the irradiated SPEC could be due to the loss of the cytoskeleton component that helps maintain the structure of the construct, while also losing adhesions between the endothelial cells. When Hiroloxamer was added prior to irradiation, however, there were still lacy endothelial networks that spanned throughout the construct embedded in a dense F-actin network, similar to what was observed in the control non-irradiated SPEC. These results indicate that Hiroloxamer helps preserve the actin cytoskeleton, while also helping maintain cell-cell junctions following exposure to ionizing radiation.

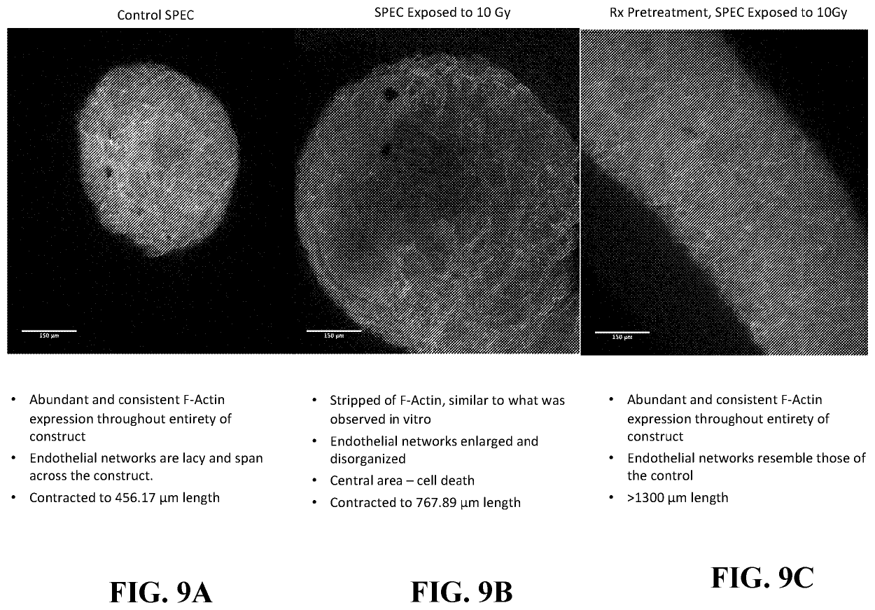

FIGS. 9A-9C show fluorescent images that can demonstrate the effect of pretreatment with Hiorloxamer on F-actin and endothelial networks. SPEC Control: EC Networks are primitive, capillary-like networks. Control SPEC (6): 73.655 μm with 3.51 μm step size (22 steps). No Rx, XRT SPEC (3): 76.027 μm with 4 μm step size (28 steps). Rx Pretreat, Full Exposure SPEC (1): 88.031 μm with 4 μm step size (23 steps).

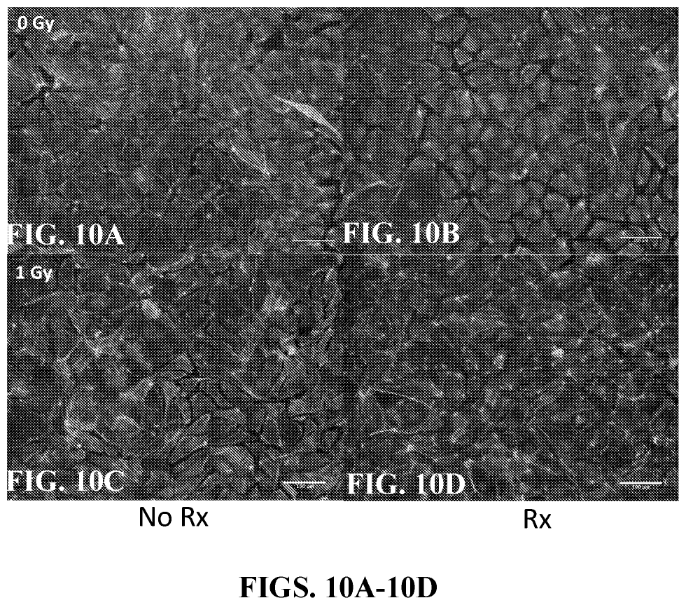

FIGS. 10A-10D show fluorescent images that can demonstrate the effect of pretreatment with Hiroloxamer on cells exposed to 1 Gy of radiation.

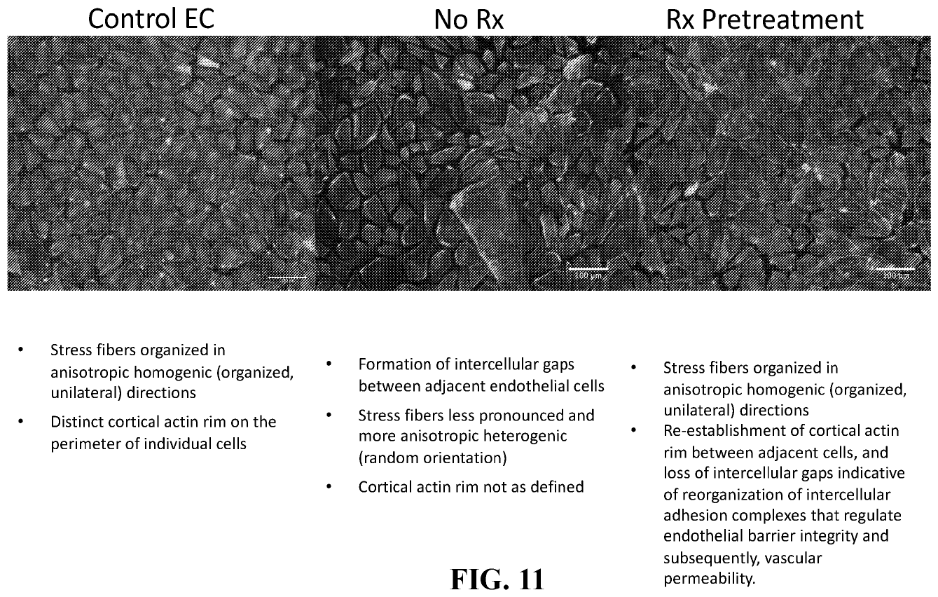

FIG. 11 shows fluorescent images that can demonstrate that 2 Gy exposure can lead to intracellular gap formation, which can be mitigated with pretreatment of Hiroloxamer. Stress fibers are required for inducing cell contraction, and dramatically influence the rate and size of the inter-endothelial gaps that form as cells retract from their borders (Pasain). Direct association of the actin cytoskeleton with cell adhesion proteins is essential to barrier function. Tight junctions and adherens junctions connect adjacent cells and regulate paracellular permeability. No Rx: Formation of intercellular gaps indicative of the loss of cell-cell junctions, which causes the depolymerization of F-actin in Ecs.

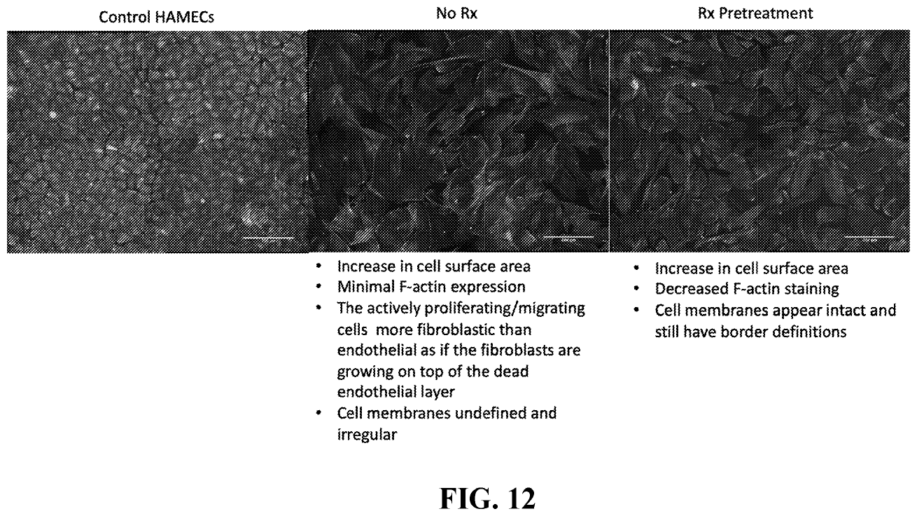

FIG. 12 shows show fluorescent images that can demonstrate the effect of pretreatment with Hiroloxamer on cells exposed to 50 Gy of radiation.

### Example 6—Effect of Hiroloxamer Pretreatment In Vivo

Discussed and demonstrated in at least this Example are the effect of pretreatment with Hiroloxamer on the radiation damage to lung tissue. Control, healthy lung tissue for comparison to the results presented herein is discussed and shown in Cho et al., Korean J Physiol Pharmacol. 2013. August: 17(4):267-274 and Almeida et al (2013) PLoS ONE 8(1): e53628. A reference histological presentation of the progression of radiation-induced lung damage is discussed in Sun et al. J. Radiat Res. 2014. July:55(4):683-689 and Almeida et al (2013) PLoS ONE 8(1):e53628. Cho et al. also discusses and provides a histological presentation of changes in macroscopic features of the lung after radiation exposure, particularly thickening of the vasculature lining around bronchioles, thickening of the fine lung parenchyma, inflammatory infiltrate, and deposition of fibrinous exudate, and edema and thickening of the vasculature.

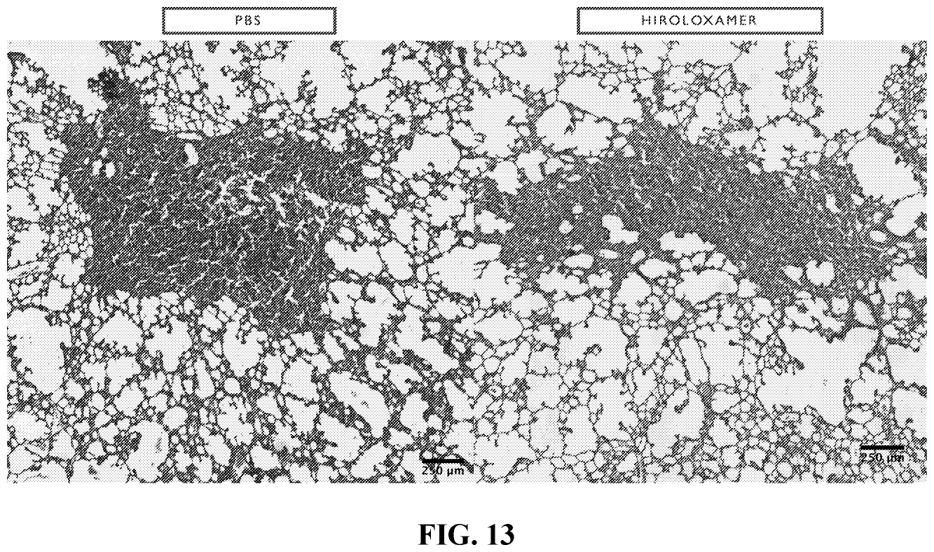

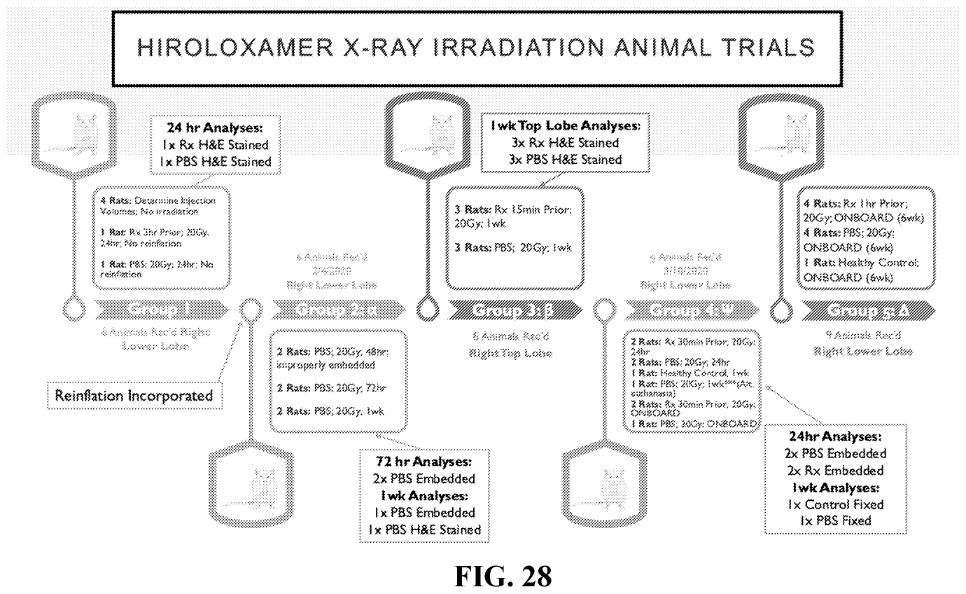

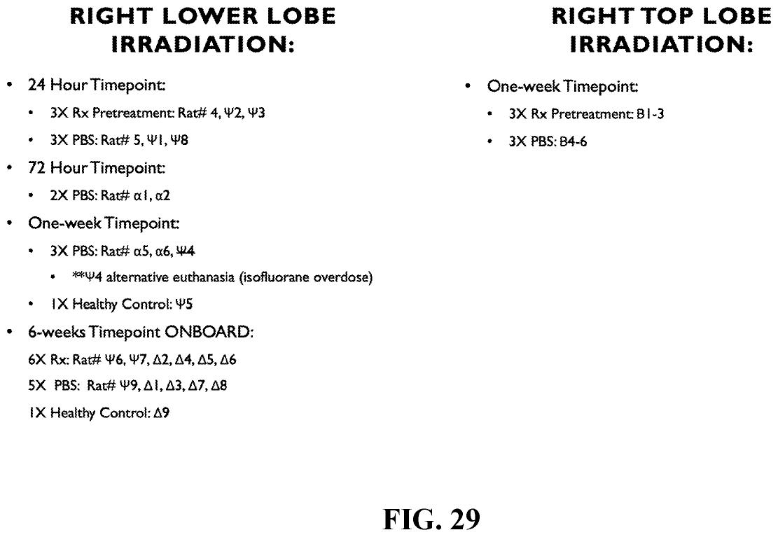

FIG. 28 shows an irradiation protocol for these experiments whose results are shown in FIGS. 13-27. FIG. 29 shows a summary of the animal groups used in these experiments.

FIG. 13 shows lung histology of PBS or Hiroloxamer treated rats that can demonstrate the effect of 200 mg/kg Hiroloxamer treatment 3 hours prior to 20 Gy irradiation. Lung samples for histology were taken about 24 hours post irradiation.

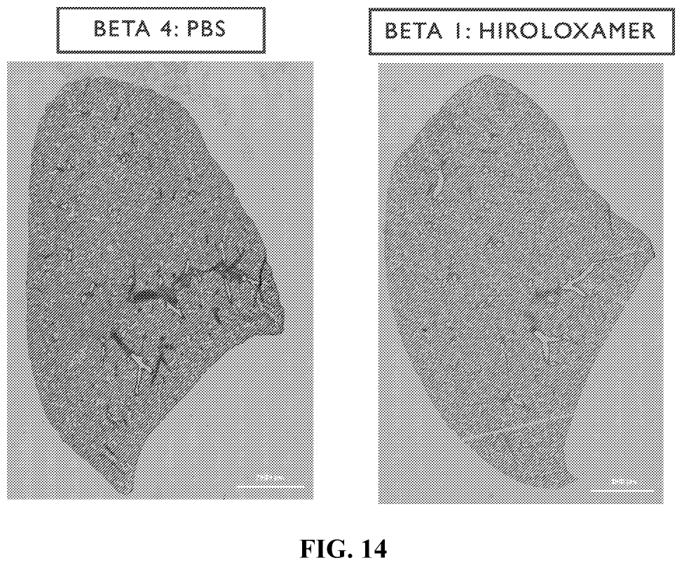

FIG. 14 shows lung histology of PBS or Hiroloxamer treated rats that can demonstrate the effect of a 200 mg/kg Hiroloxamer treatment administered 15 min prior to exposure to 20 Gy of irradiation.

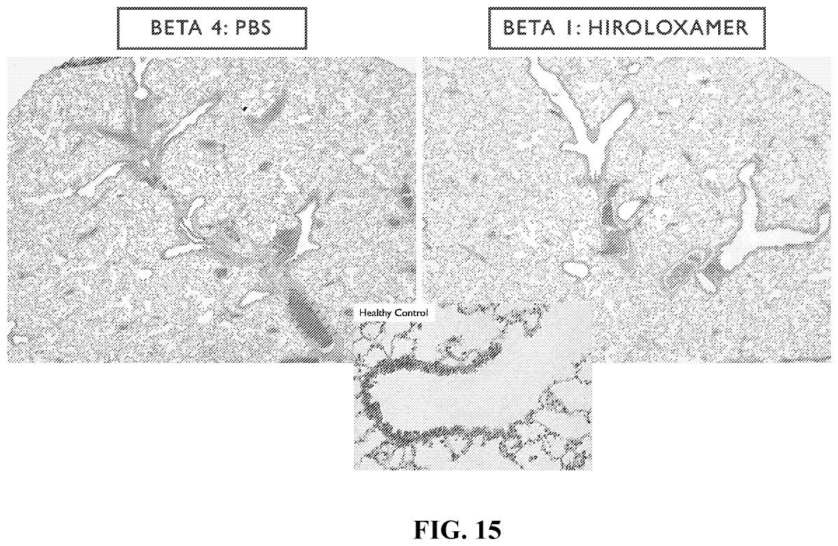

FIG. 15 shows lung histology of PBS or Hiroloxamer treated rats that can demonstrate the effect of a 200 mg/kg Hiroloxamer treatment administered 15 min prior to exposure to 20 Gy of irradiation. The histology reference of the healthy control was obtained from http://histology.oucreate.com/Captions/Respiratory/109.ling.mammal/109.bronchiole.cl.40.L htm#click.

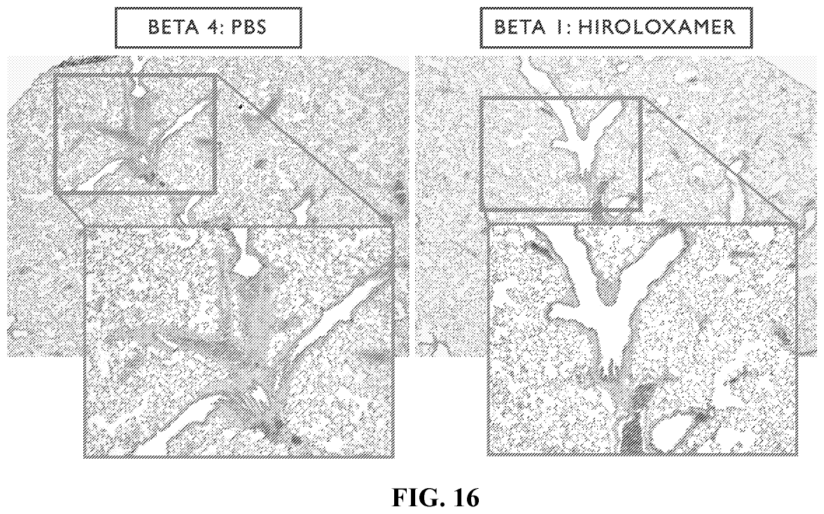

FIG. 16 shows lung histology of PBS or Hiroloxamer treated rats that can demonstrate the effect of a 200 mg/kg Hiroloxamer treatment administered 15 min prior to exposure to 20 Gy of irradiation.

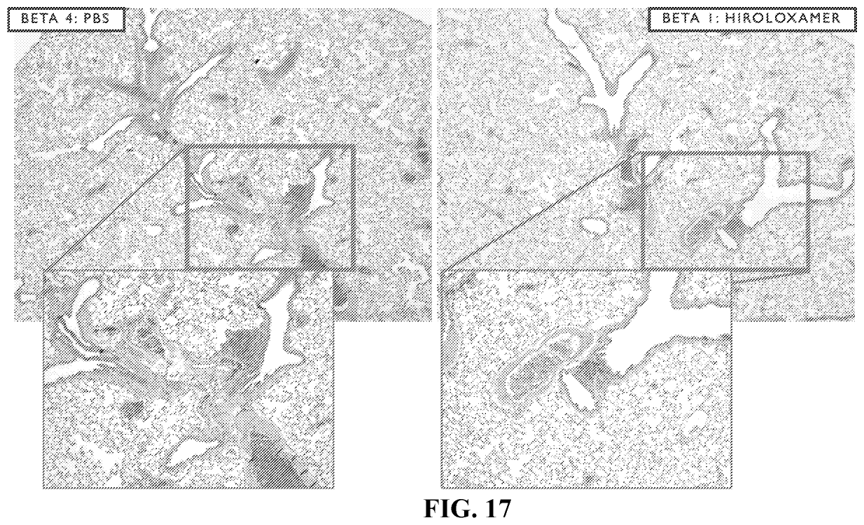

FIG. 17 shows lung histology of PBS or Hiroloxamer treated rats that can demonstrate the effect of a 200 mg/kg Hiroloxamer treatment administered 15 min prior to exposure to 20 Gy of irradiation.

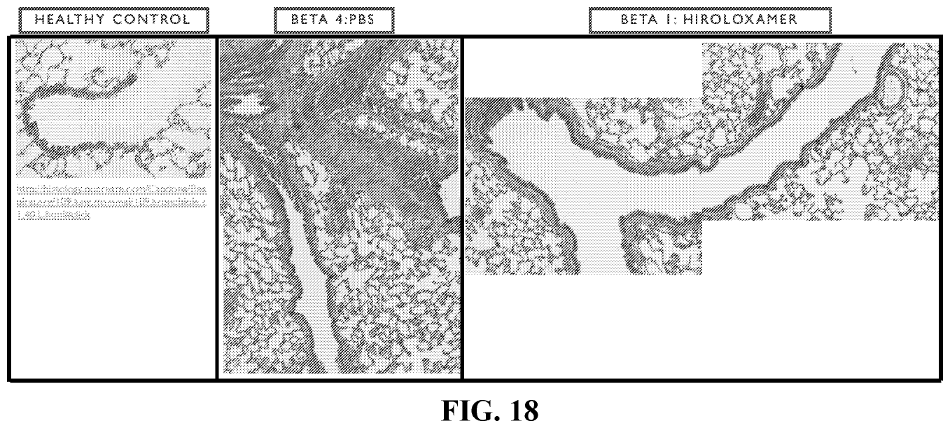

FIG. 18 shows lung histology of PBS or Hiroloxamer treated rats that can demonstrate that Hiroloxamer can conserve oxygen pathways in an irradiated lung at bronchiole branches. The histology reference of the healthy control was obtained from http://histology.oucreate.com/Captions/Respiratory/109.ling.mammal/109.bronchiole.cl.40.L htm#click.

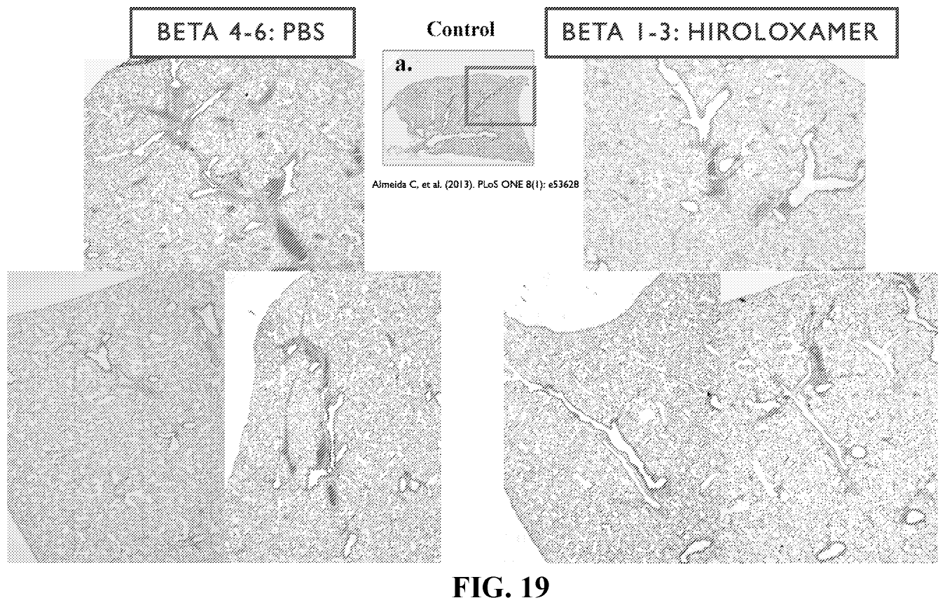

FIG. 19 shows lung histology of PBS or Hiroloxamer treated rats that can demonstrate the effect of a 200 mg/kg Hiroloxamer treatment administered 15 min prior to exposure to 20 Gy of irradiation.

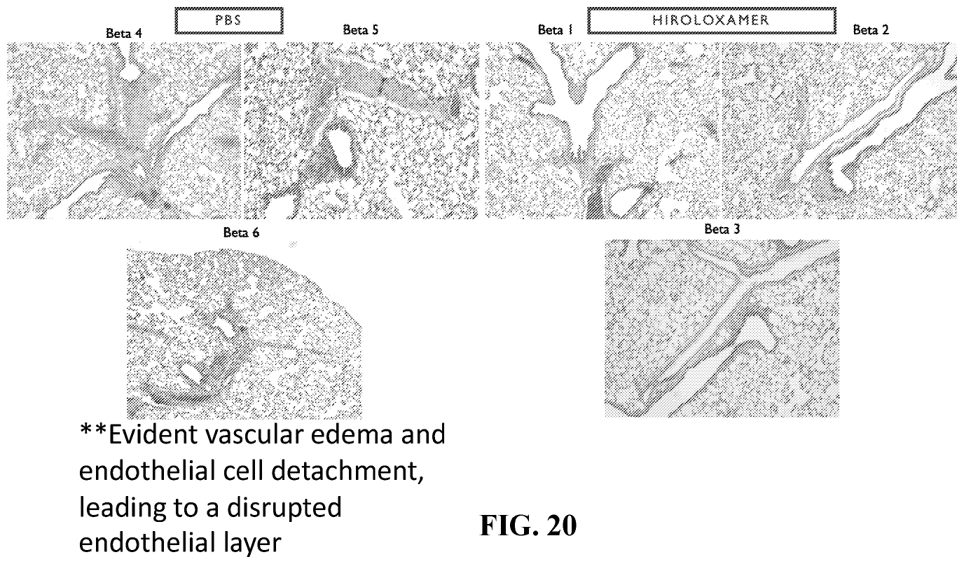

FIG. 20 shows lung histology of PBS or Hiroloxamer treated rats that can demonstrate that Hiroloxamer attenuates inflammation of bronchioles via protection of the vasculature.

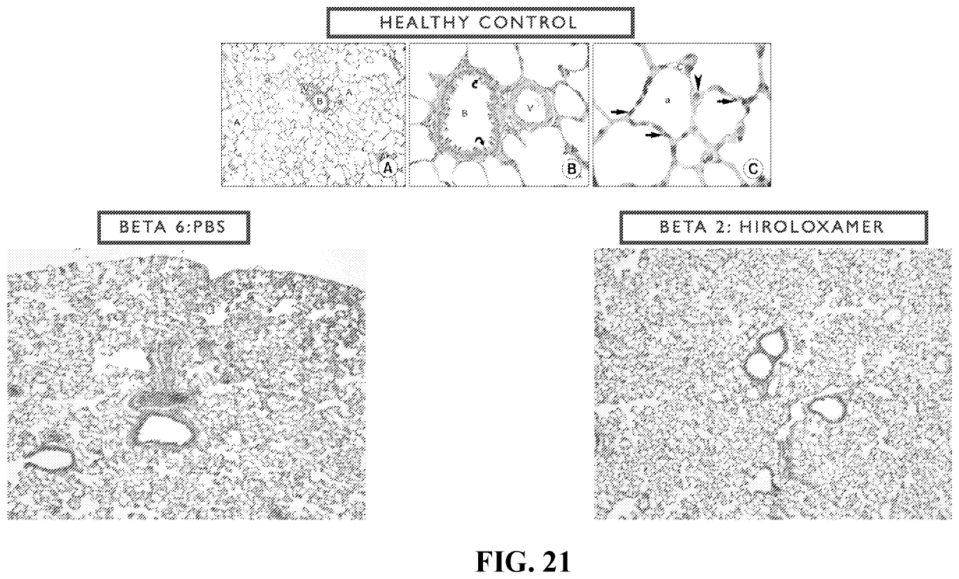

FIG. 21 shows lung histology of PBS and Hiroloxamer treated rats and a healthy control that can demonstrate that Hiroloxamer can conserve blood vessel (V) and bronchiole (B) architectures.

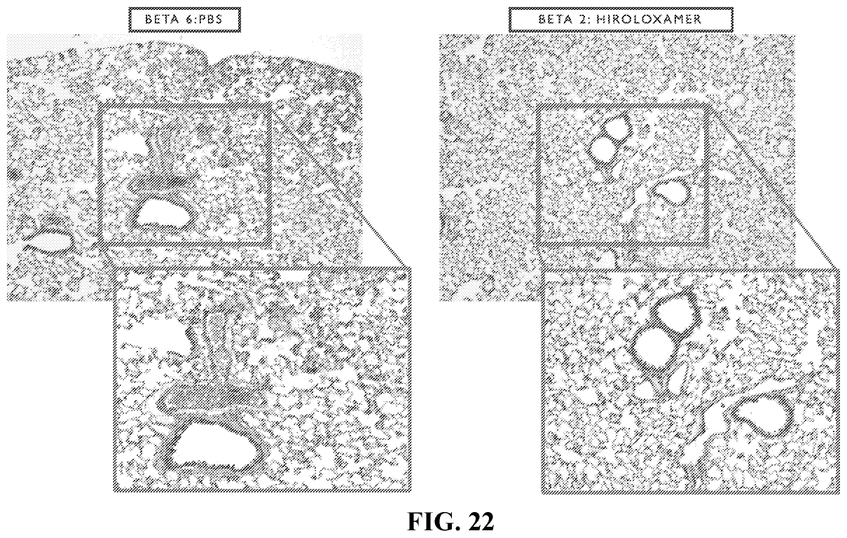

FIG. 22 shows lung histology of PBS and Hiroloxamer treated rats that can demonstrate that Hiroloxamer can conserve blood vessel (V) and bronchiole (B) architectures. FIG. 22 are higher magnification images of those shown in FIG. 21.

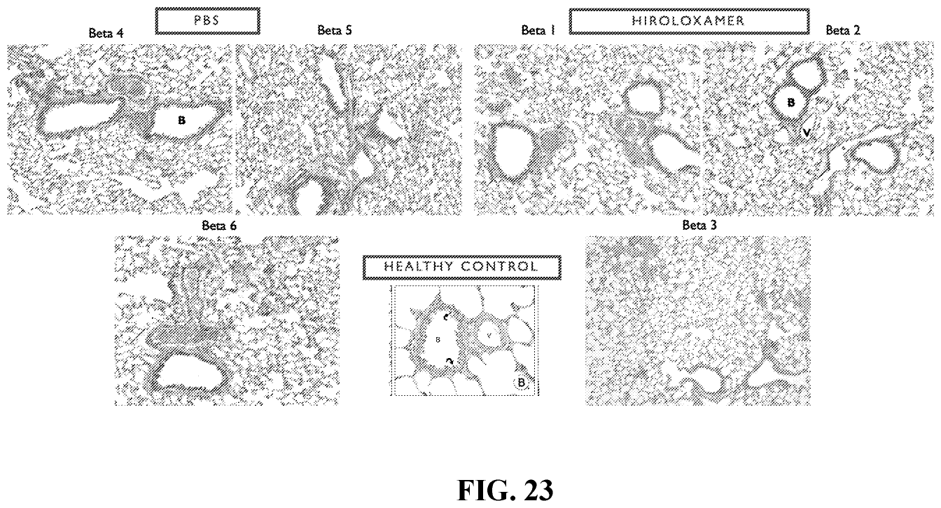

FIG. 23 shows lung histology of PBS and Hiroloxamer treated rats that can demonstrate that Hiroloxamer can conserve blood vessel (V) and bronchiole (B) architectures. Also shown is a healthy control.

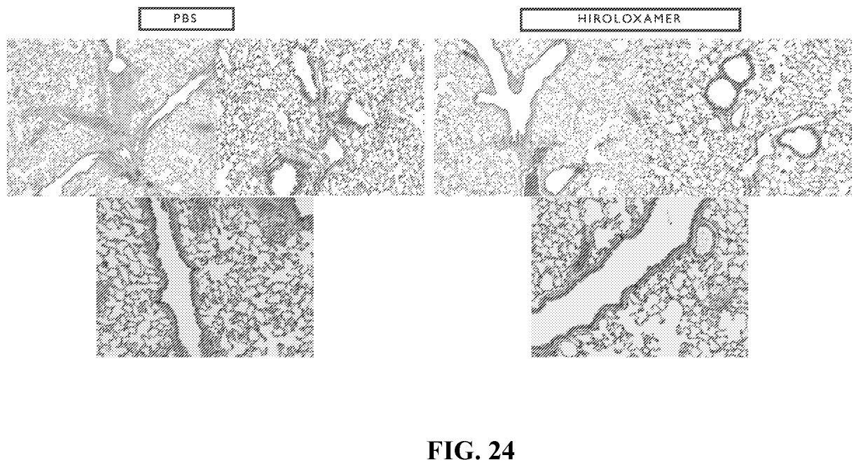

FIG. 24 shows lung histology of PBS and Hiroloxamer that can demonstrate that Hiroloxamer can attenuate early radiation-induced damage in the lungs.

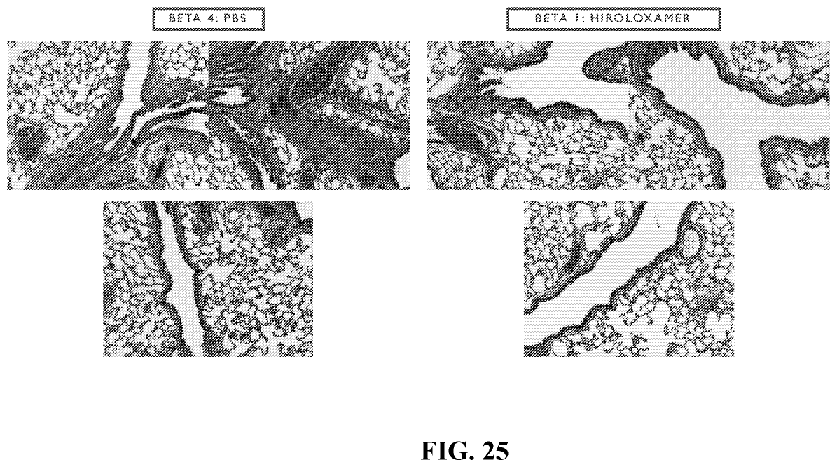

FIG. 25 shows lung histology of PBS and Hiroloxamer treated rats that can demonstrate that Hiroloxamer can conserve perivasculature of irradiated lungs.

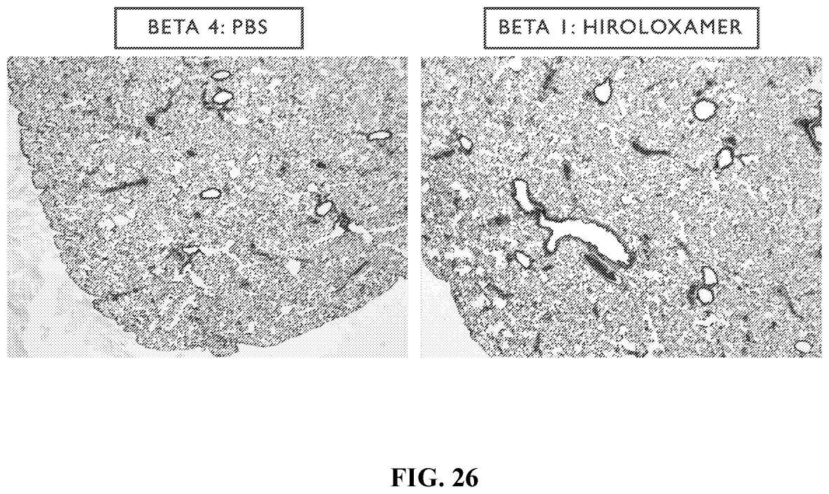

FIG. 26 shows lung histology of PBS and Hiroloxamer treated rats that can demonstrate that Hiroloxamer can conserve perivasculature of irradiated lungs.

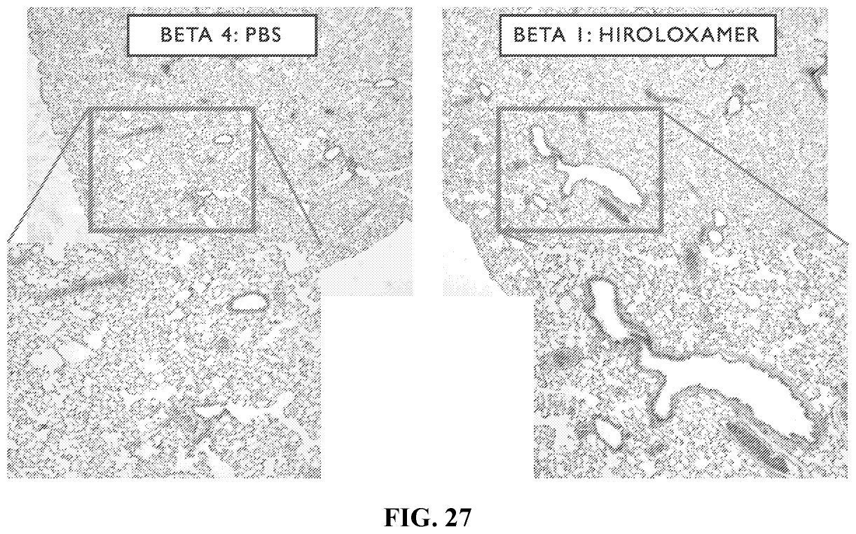

FIG. 27 shows lung histology of PBS and Hiroloxamer treated rats that can demonstrate that Hiroloxamer can conserve perivasculature of irradiated lungs. FIG. 27 is a higher magnification of the images shown in FIG. 26.

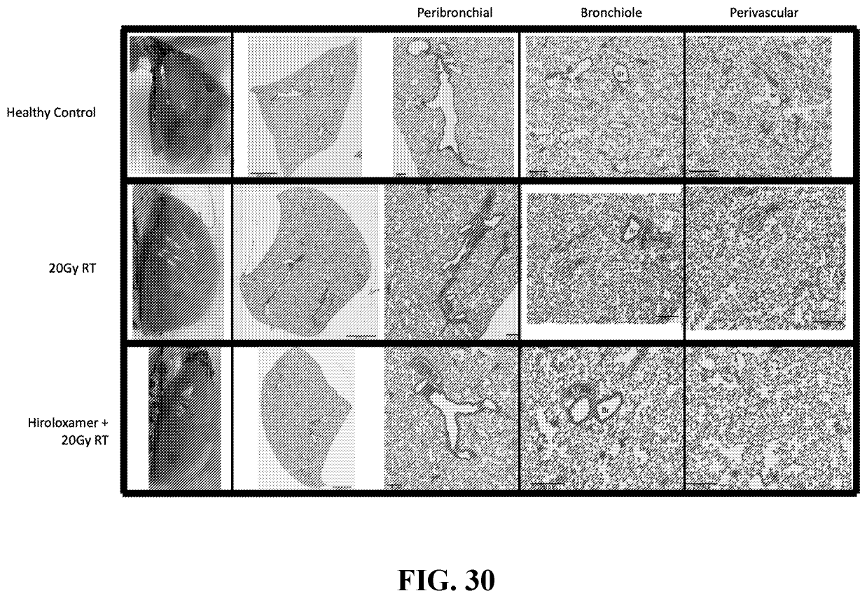

FIG. 30 shows results 1 week post-irradiation (20 Gy). FIG. 30 shows a panel of representative photomicrographs of Hematoxylin & Eosin (H&E)-stained lung sections from Control, Radiation (RT), and Hiroloxamer+Radiation one week post-irradiation (Scale bar=250 μm). The control group had normal (clear) lumen of lung bronchi (“Br”) and normal lumen of air alveoli. The RT group had evident inflammatory infiltrate throughout the peribronchial, almost to occlusion, as well as collapsed blood vessels and capillaries, thickening of intra-alveolar septa, and cellular infiltrate surrounding bronchi structures. The Hiroloxamer+RT group had some minor inflammation around major vessels in the peribronchial, but alveoli and perivascular appeared normal.

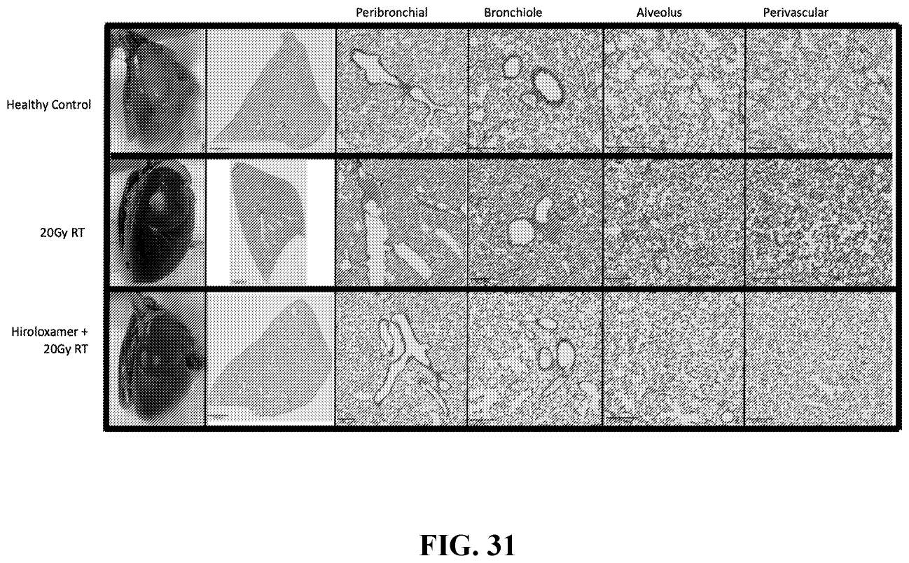

FIG. 31 shows results 6 week post-irradiation (20 Gy) that can demonstrate that Hioloxamer can preserve healthy lung tissue in a partial volume rat lung X-ray irradiation model. FIG. 31 shows representative photomicrographs of Hematoxylin & Eosin (H&E)-stained right lung lower lobe sections from Control, Radiation (RT), and Hiroloxamer+Radiation six weeks post-irradiation (Scale bar=250 μm). The control group had normal (clear) lumen of lung bronchi (“Br”) and normal lumen of air alveoli. The RT group had marked interstitial edema, congested blood vessel and capillaries (“V”), increase in alveolar septal thickness, and dense inflammatory infiltrate throughout the entire lobe that expanded distally into the right upper lung lobe. The Hiroloxamer+RT group had some inflammatory infiltrate surrounding some bronchioles located near some minor alveoli thickening, blood vessels remained intact with minimal surrounding inflammatory infiltrate.

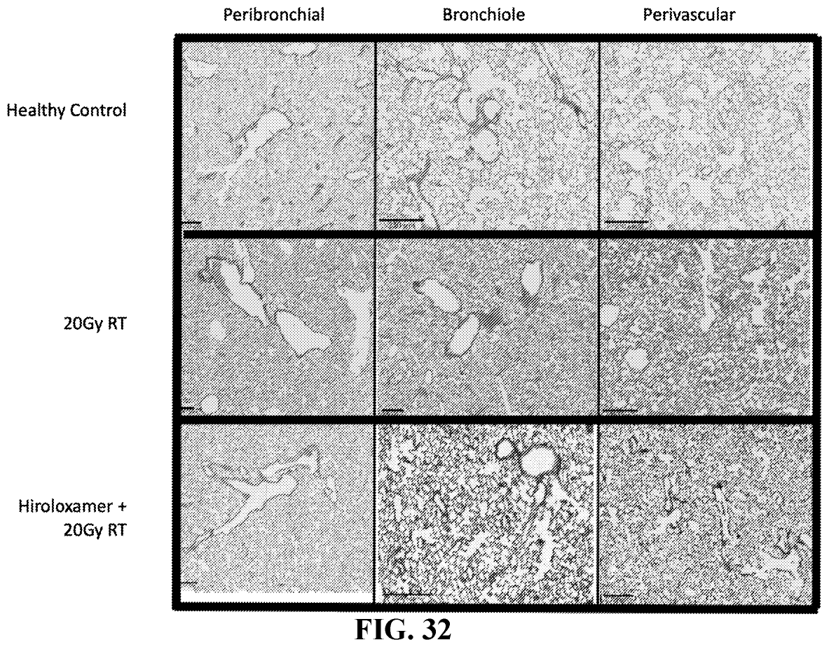

FIG. 32 shows representative photomicrographs of Picrosirius Red stained right lung lower lobe section 6 weeks post-irradiation with 20Gy X-rays (scale bar=250 microns).

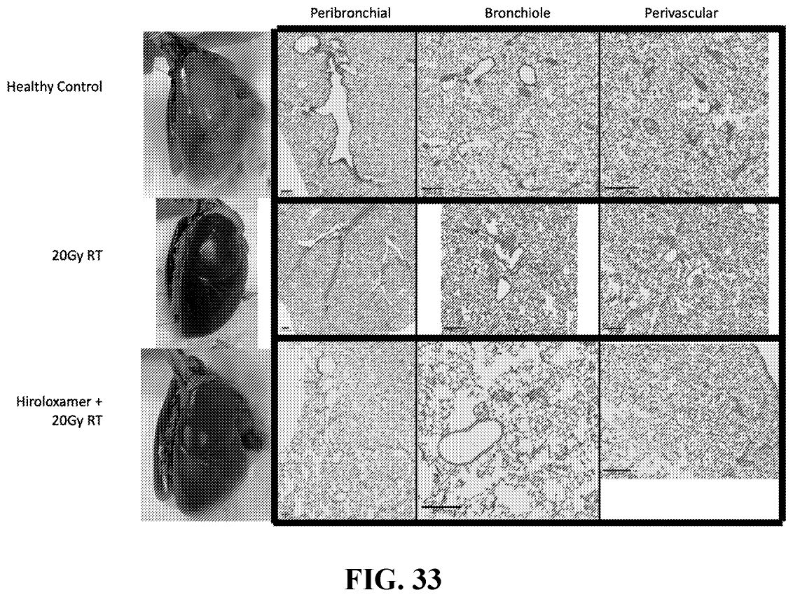

FIG. 33 shows representative photomicrographs of hematoxylin & Eosin (H&E)-stained right lung top lobe sections from Control, Radiation (RT), and Hiroloxamer+Radiation six weeks post-irradiation (Scale bar=250 microns). Radiation-induced lung damage extended from the right lower lung lobe where radiation was exposed to the distal right upper lung lobe in the 20 Gy RT group only.

The pharmacokinetics showed predominately renal clearance and were calculated as follows: Clearance=7744 mL/hr. Cₘₐₓ=0.42 mg/hr. Tₘₐₓ=30.49 hr, T1/2=4.77 hr, Mean Residence Time (MRT)=34.06 hr. Therapeutic Window: 100-150 mg/kg for 1 hour loading dose, followed by 20-40 mg/kg per hour for maintenance. Duration of action: t_{1/2}=4.77 hours (plasma half-life is about 4.5 hours after infusion has ended). Cₘₐₓ=maximum (peak) plasma drug concentration Tₘₐₓ=time to reach maximal (peak) plasma concentration following drug administration. There were no differences observed among male and female patients for any pharmacokinetic parameters across any of the subgroups.

### Example 7—Hiroloxamer can Prevent Radiation-Induced Pneumonitis and Radiation Toxicity

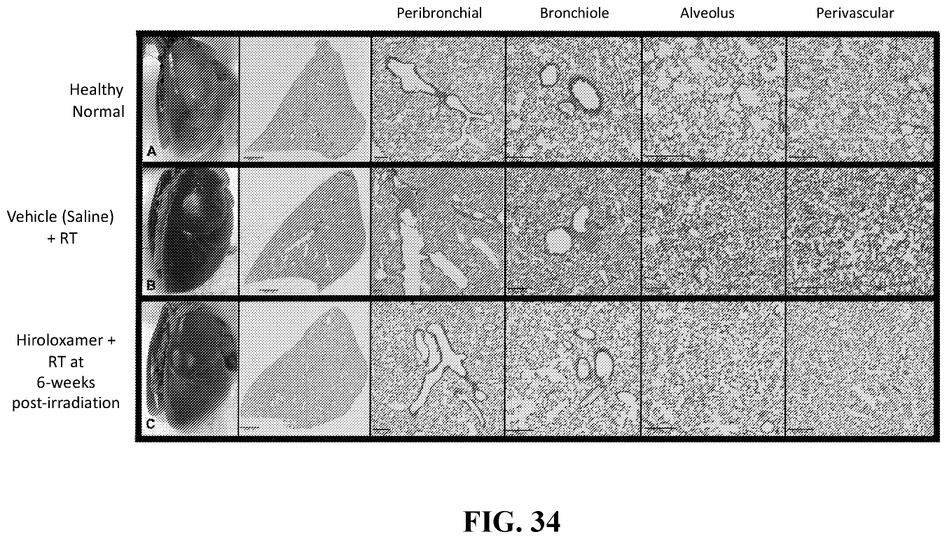

FIG. 34 shows representative images and photomicrographs that can demonstrate that Hiroloxamer prevents the onset of acute pneumonitis in a rat model of radiation-induced pneumonitis. Exposure of SD rats to a single fraction of 20Gy x-ray irradiation is a prescribed dose for radiation-induced pneumonitis (Ghita). Rats were anesthetized and restrained in a custom jig designed to expose only 6 mm of the right center lung lobe to irradiation, while the rest of the animal remained shielded through a lead shield. To investigate the onset and progression of pneumonitis, proximal and distal tissues to the radiation site were stained with Hematoxylin and Eosin (H&E) and evaluated to assess integrity of the major lung structures at 6 weeks post-irradiation. Representative photomicrographs of the right lung lower lobe sections from Healthy Normal, Vehicle (Saline)+RT, and Hiroloxamer+RT at 6-weeks post-irradiation. Vehicle (saline)+RT control animals developed acute pneumonitis within 6 weeks post-irradiation, as evidenced by capillary leak, marked interstitial edema, congested blood vessels and capillaries, increase in alveolar thickness, and dense inflammatory infiltrate that propagated distally to adjacent lung lobes and to the contralateral left lung. Animals treated with Hiroloxamer prior to irradiation (Hiroloxamer+RT) had clear lumen of peribronchial and bronchi with alveolar septum appearing normal as in the healthy control animals (Healthy Normal); n=3 for each group.

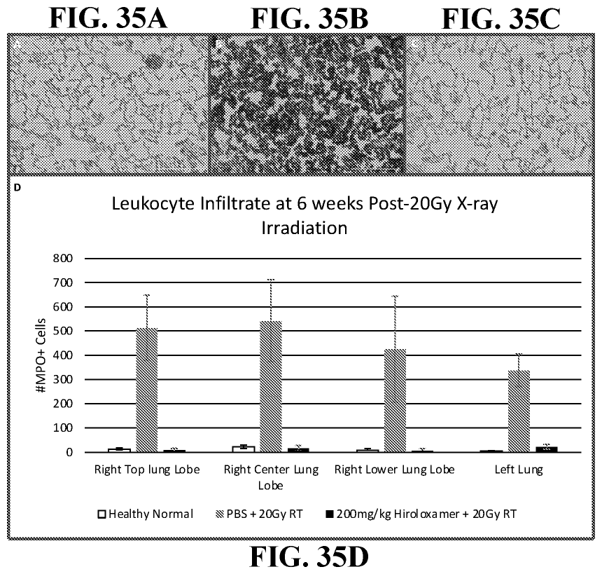

FIGS. 35A-35D shows representative photomicrographs (FIGS. 35A-35C) and a graph (FIG. 35D) that demonstrate an MPO Analysis of Leukocyte Infiltrate. Controlled neutrophil degranulation and myeloperoxidase (MPO) release at a site of damage is necessary for effective wound healing. However, over exuberant degranulation exaggerates the inflammatory response and can lead to tissue damage even in the absence of infection. Levels of MPO activity are indicative of the state of inflammation and oxidative stress in tissues. SD rats were irradiated as described previously with a single fraction of 20Gy X-ray to the right center lung lobe through a 6 mm hole in a lead shield to model radiation-induced pneumonitis. Sections from the three right lung lobes, as well as the contralateral left lung lobe were paraffin-embedded, sectioned to 5 μm, and stained with Hanker-Yates Peroxidase Leukocyte kit (Sigma Aldrich) for analyses. Stained sections were imaged using a LionHeartFX automated microscope, and MPO+ cell counts automated via thresholding in Gen5 software. Five high powered fields were analyzed for each animal with three animals per group. Representative MPO-stained right lower lung lobe sections of (FIG. 35A) Healthy control, (FIG. 35B) vehicle (Saline)+RT, (FIG. 35C) 200 mg/kg Hiroloxamer+RT. Tissue damage was observed in the control saline+RT animals, that had significantly elevated levels of MPO in all lung lobes compared to the Hiroloxamer+RT and healthy normal (FIG. 35D). Statistical comparison (ANOVA followed by Tukey) of healthy normal, vehicle (Saline)+RT, and Hiroloxamer+RT shows no difference between healthy normal and Hiroloxamer+RT, while there is a significant statistical difference between vehicle (Saline)+RT and healthy normal (p<0.001), and vehicle (Saline)+RT and Hiroloxamer+RT (p<0.001).

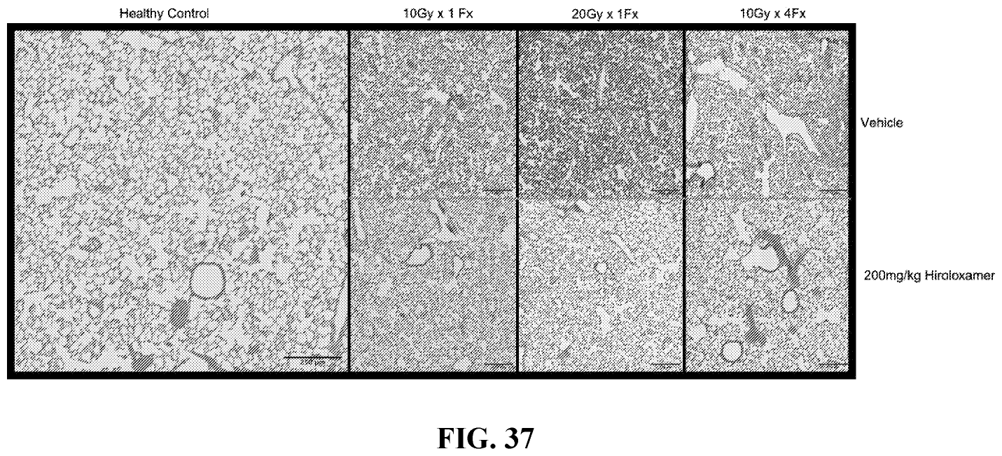

FIG. 37 shows representative photomicrographic images that can demonstrate that Hiroloxamer protects healthy tissue from radiation toxicity independent of irradiation dose. Animals were exposed to either 10Gy×1 fraction (fx), 20Gy×1fx, or 10Gy×4fx of X-ray irradiation delivered every 2-3 days over 2 weeks, to an 8 mm area of the right lung. All animals were sacrificed at 6-weeks post-irradiation and stained with H&E (5 μm sections). Animals treated with vehicle (top row of figures) had evident radiation-induced lung injury of varying degrees based on dose. 20Gy×1fx had the most significant damage, evidenced by edema, inflammatory infiltrate, and congestion of the lung parenchyma that were not localized to the irradiated but spread to all lung lobes bilaterally. These histological findings are indicative of the onset of radiation-induced acute pneumonitis. 10Gy×1fx and 10Gy×4fx had less damage than the 20Gy×1fx, which aligns with the classical theory that healthy tissue toxicity is dependent on the dose per fraction rather than the total dose delivered. Animals pre-treated with 200 mg/kg Hiroloxamer only had damage localized to the targeted irradiated area, with healthy tissue protected from toxicities in all radiotherapy regimens assessed.

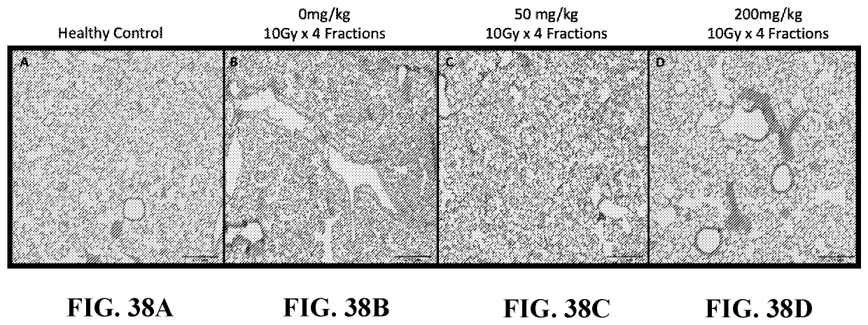

FIGS. 38A-38D shows representative photomicrographic images that can demonstrate that Hiroloxamer protects healthy tissue from radiation toxicity in a dose-dependent manner. Animals were exposed to 10Gy×4 fractions of X-ray irradiation, delivered every 2-3 days over 2 weeks, to an 8 mm area of the right lung. Animals treated with 200 mg/kg Hiroloxamer (FIG. 38D) prior to irradiation had normal lung appearance comparable to healthy control (FIG. 38A). Animals treated with 50 mg/kg Hiroloxamer (FIG. 38C) had evident cellular infiltrate and subsequent thickening of the alveoli that was not as severe as the vehicle (PBS)-treated animals (FIG. 38B), but not adequate protection compared to the 200 mg/kg dose.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A method of preventing damage to non-insulted cells in a subject during and/or after insult exposure comprising:

administering a first amount of poloxamer 188 (P188) or a pharmaceutical formulation thereof to a subject prior to exposure of one or more regions of the subject to an insult; and

exposing one or more regions of the subject to an insult after the blood concentration of P188 reaches between 1 and 5 mg/mL.

2. The method of aspect 1, wherein the first amount of P188 is administered intravenously.

3. The method of any one of aspects 1-2, further comprising administering an n^{th} amount of P188 or a pharmaceutical formulation thereof to the subject after administering the first amount of P188 and before exposing one or more regions of the subject to the insult, wherein n is 2 or more.

4. The method of any one of aspects 1-3, wherein the first amount, the n^{th} amount, or all the amounts together of P188 is/are an amount effective to raise the blood concentration of P188 to 1 to 5 mg/mL within 1 to 48 hours.

5. The method of any one of aspects 1-4, wherein the first amount or the n^{th} amount of P188 is administered as a continuous infusion over a period of time.

6. The method of any one of aspects 1-5, wherein the first amount or one or more of the n^{th} amounts of P188 is administered as a bolus amount.

7. The method of any one of aspects 1-2 and 4-6, wherein only the first amount is administered and wherein the first amount is effective to raise the blood concentration of P188 to about 1 to about 5 mg/mL within 1 to 48 hours.

8. The method of any one of aspects 1-7, wherein the subject does not receive any amount of P188 for at least 14 hours after receiving the first or n^{th} amount of P188.

9. The method of any one of aspects 1-8, wherein P188 administration is discontinued immediately after exposing one or more regions of the subject to the insult.

10. The method of any one of aspects 1-9, wherein exposing one or more regions of the subject to the insult occurs about 1 to about 48 hours after administering the first or the n^{th} amount of P188.

11. The method of any one of aspects 1-10, wherein the insult is effective to kill one or more cancerous cells with in the one or more regions of the subject.

12. The method of any one of aspects 1-11, wherein the non-insulted cells are non-cancerous cells.

13. The method of any one of aspects 1-12, wherein the non-insulted cells are endothelial cells.

14. The method of any one of aspects 1-13, wherein the insult is a mechanical insult, a chemical insult, a biological insult, an energetic insult, a physiologic insult, or a combination thereof.

15. The method of any one of aspects 1-14, wherein the insult is ionizing radiation.

16. A method preventing damage to non-insulted cells in a subject during and/or after insult exposure comprising:

administering an amount of poloxamer 188 (P188) or a pharmaceutical formulation thereof to a subject prior to exposure of one or more regions of the subject to an insult, wherein the amount ranges from about 10 to 150 mg/kg; and

exposing one or more regions of the subject to the insult within 0-48 hours after administering the amount of P188 or pharmaceutical formulation thereof.

17. The method of aspect 16, wherein the amount of P188 or pharmaceutical formulation thereof is administered intravenously.

18. The method of any one of aspects 16-17, wherein the administration of P188 or pharmaceutical formulation thereof is discontinued immediately prior to or immediately after exposing one or more regions of the subject to the insult.

19. The method of any one of aspects 16-18, wherein the subject does not receive any amount of P188 or pharmaceutical formulation thereof for at least 14 hours after administering the amount of P188 or pharmaceutical formulation thereof or after discontinuing the administration of P188 or pharmaceutical formulation thereof.

20. The method of any one of aspects 16-18, wherein the insult is a mechanical insult, a chemical insult, a biological insult, an energetic insult, a physiologic insult, or a combination thereof.

21. The method of any one of aspects 16-20, wherein the insult is ionizing radiation.

22. A kit comprising:

an amount of P188 or a pharmaceutical formulation thereof, wherein the amount of P188 or pharmaceutical formulation is effective to prevent damage to non-insulted cells in a subject when one or more regions of the subject are exposed to an insult; and

instructions fixed in a tangible medium of expression directing administration of the amount of the amount of P188 or pharmaceutical formulation thereof to a subject 0-48 hours prior to exposure, 0-48 hours after exposure, or both of one or more regions of the subject to the insult.

23. The kit of aspect 22, wherein the insult is a mechanical insult, a chemical insult, a biological insult, an energetic insult, a physiologic insult, or a combination thereof.

24. The kit of any one of aspects 22-23, wherein the insult is ionizing radiation.

25. The kit of any one of aspects 22-24, wherein the instructions further direct for discontinuation of administration of the amount of P188 or pharmaceutical formulation thereof to the subject immediately prior to or immediately after exposure of one or more regions of the subject to the insult.

26. The kit of any one of aspects 22-25, wherein the amount of P188 or pharmaceutical formulation thereof is effective to achieve a blood concentration of about 1 to about 5 mg/mL in the subject within 0-48 hours.

27. A method of protecting non-insulted cells after exposure of a subject to an insult comprising:
a) administering an amount of P188 or a formulation thereof to a subject prior to exposure to an insult;
b) administering an amount of P188 or a formulation thereof to a subject immediately following exposure to an insult;
c) administering an amount of P188 or a formulation thereof to a subject during an insult; or
d) a combination thereof.

28. The method of aspect 27, wherein the amount is effective increase the blood concentration of P188 in the subject to between 1 mg/mL and 5 mg/mL.

29. The method of any one of aspects 27-28, wherein the amount is effective to coat one or more non-insulted cells.

30. The method of any one of aspects 27-29, wherein the amount is effective to reduce or prevent oxidative damage to one or more non-insulted cells.

31. The method of any one of aspects 27-30, wherein the amount is effective to reduce or prevent inflammatory damage to one or more non-insulted cells.

32. The method of any one of aspects 27-31, wherein administering occurs 0-48 hours prior to exposure to the insult.

33. The method of any one of aspects 27-32, wherein administering occurs 0-48 hours or 0-24 hours post exposure to the insult.

34. The method of any one of aspects 27-32, wherein the insult is a mechanical insult, a chemical insult, a biological insult, an energetic insult, a physiologic insult, or a combination thereof.

35. The method of any one of aspects 27-34, wherein the insult is ionizing radiation.

36. A method of preventing insult-induced pneumonitis in a subject, the method comprising:
a) administering an amount of P188 or a formulation thereof to a subject prior to exposure to an insult;
b) administering an amount of P188 or a formulation thereof to a subject immediately following exposure to an insult;
c) administering an amount of P188 or a formulation thereof to a subject during an insult; or
d) a combination thereof.

37. The method of aspect 36, wherein the amount is effective increase the blood concentration of P188 in the subject to between 1 mg/mL and 5 mg/mL.

38. The method of any one of aspects 36-37, wherein the amount is effective to coat one or more non-insulted cells.

39. The method of any one of aspects 36-38, wherein the amount is effective to reduce or prevent oxidative damage to one or more non-insulted cells.

40. The method of any one of aspects 36-39, wherein the amount is effective to reduce or prevent inflammatory damage to one or more non-insulted cells.

41. The method of any one of aspects 36-40, wherein administering occurs 0-48 hours prior to exposure to the insult.

42, The method of any one of aspects 36-41, wherein administering occurs 0-24 hours or 0-48 hours post exposure to the insult.

43. The method of any one of aspects 36-42, wherein the insult is a mechanical insult, a chemical insult, a biological insult, an energetic insult, a physiologic insult, or a combination thereof.

44. The method of any one of aspects 36-43, wherein the insult is ionizing radiation.

45. A method of treating a disease or disorder in a subject in need thereof, the method comprising:
a) administering an amount of P188 or a formulation thereof to a subject prior to exposure to an insult;
b) administering an amount of P188 or a formulation thereof to a subject immediately following exposure to an insult;
c) administering an amount of P188 or a formulation thereof to a subject during an insult; or
d) a combination thereof.

46. The method of aspect 45, wherein the amount is effective increase the blood concentration of P188 in the subject to between 1 mg/mL and 5 mg/mL.

47. The method of any one of aspects 45-46, wherein the amount is effective to coat one or more non-insulted cells.

48. The method of any one of aspects 45-47, wherein the amount is effective to reduce or prevent oxidative damage to one or more non-insulted cells.

49. The method of any one of aspects 45-48, wherein the amount is effective to reduce or prevent inflammatory damage to one or more non-insulted cells.

50. The method of any one of aspects 45-49, wherein administering occurs 0-48 hours prior to exposure to the insult.

51. The method of any one of aspects 45-50, wherein administering occurs 0-24 hours or 0-48 hours post exposure to the insult.

52. The method of any one of aspects 45-51, wherein the insult is a mechanical insult, a chemical insult, a biological insult, an energetic insult, a physiologic insult, or a combination thereof.

53. The method of aspect 52, wherein the insult is an ischemic event, optionally a stroke or myocardial infarction.

1-53. (canceled)

54. A method of preventing damage to non-insulted cells in a subject during and/or after insult exposure comprising:
administering an amount of poloxamer 188 (P188) or a pharmaceutical formulation thereof to a subject prior to exposure of one or more regions of the subject to an insult, wherein the amount ranges from about 10 to 150 mg/kg; and
exposing one or more regions of the subject to the insult within 0-48 hours after administering the amount of P188 of pharmaceutical formulation thereof,
wherein the subject does not receive any amount of P188 or pharmaceutical formulation thereof for at least 14 hours after administering the amount of P188 or pharmaceutical formulation thereof or after discontinuing the administration of P188 or pharmaceutical formulation thereof.

55. The method of claim 54, wherein the insult is a mechanical insult, a chemical insult, a biological insult, an energetic insult, a physiologic insult, or any combination thereof.

56. The method of claim 54, wherein the insult is ionizing radiation.

57. The method of claim 54, wherein the amount of P188 is administered intravenously.

58. The method of claim 54, wherein the amount of P188 is administered as a bolus amount or as a continuous infusion.

59. The method of claim 54, wherein the amount of P188 is an amount effective to raise the blood concentration of P188 to 1 to 5 mg/mL within 1 to 48 hours.

60. The method of claim 54, wherein the insult is effective to kill one or more cancerous cells with in the one or more regions of the subject.

61. The method of claim 54, wherein the non-insulted cells are non-cancerous cells, are endothelial cells, or are both.

62. The method of claim 54, wherein the damage is oxidative damage.

63. The method of claim 54, wherein the damage is inflammatory damage.

64. The method of claim 54, wherein the insult is an ischemic event.

65. The method of claim 64, wherein the ischemic event is a stroke or myocardial infarction.

66. The method of claim 54, wherein the amount of P188 is effective to coat one or more of the non-insulted cells.
